(19) United States
(12) Patent Application Publication
(10) Pub. No.: US 2023/0248962 A1
(43) Pub. Date: Aug. 10, 2023

(54) SYSTEM, METHOD, AND APPARATUS FOR APPLYING BILATERAL TRANSCUTANEOUS ELECTRICAL STIMULATION

(71) Applicant: AVATION MEDICAL, INC., Columbus, OH (US)

(72) Inventors: Mingming Zhang, Hilliard, OH (US); Manish Vaishya, Valencia, CA (US)

(21) Appl. No.: 18/107,241

(22) Filed: Feb. 8, 2023

Related U.S. Application Data
(60) Provisional application No. 63/307,702, filed on Feb. 8, 2022.

(51) Int. Cl. A61N 1/04; A61N 1/02; A61N 1/36

(52) U.S. Cl. CPC A61N 1/0456; A61N 1/0476; A61N 1/0484; A61N 1/025; A61N 1/36031

(57) ABSTRACT

An apparatus for applying electrical stimulation to a target peripheral nerve in a subject includes a wearable structure configured to be worn on at least one of the subject's lower leg, foot, or ankle. A first stimulation electrode is mounted on the wearable structure at a first location on the wearable structure. A second stimulation electrode is mounted on the wearable structure at a second location on the wearable structure, different than the first location on the wearable structure. One or more recording electrodes are mounted on the wearable structure. The apparatus also includes a control unit for controlling the operation of the first stimulation electrode, the second stimulation electrode, and the one or more recording electrodes. The wearable is configured to position the first stimulation electrode on a medial side of the target peripheral nerve, and to position the second stimulation electrode on a lateral side of the target peripheral nerve. The control unit is also configured to use the first and second stimulation electrodes to apply electrical stimulation to the target peripheral nerve and to record physiological responses to the applied electrical stimulation using the recording electrodes.

### RELATED APPLICATION

This application claims priority from U.S. Provisional Patent Application Ser. No. 63/307,702, filed Feb. 8, 2022, the entirety of which is hereby incorporated herein by reference.

### TECHNICAL FIELD

The invention relates to a wearable electronic medical device for transcutaneous electrical stimulation of peripheral nerves for the purpose of treating one or more conditions.

### BACKGROUND

There are many known technologies that use electrical stimulation of peripheral nerves to treat conditions. Implantable stimulation technologies require surgical implantation of stimulation leads, with a pulse generator that is either surgically implanted or connected externally to wire leads. Percutaneous stimulation technologies are less invasive, but still require the stimulation electrodes to pierce the skin. While these technologies can be effective in treating certain conditions, they are less desirable due to their invasiveness and because they can require the continued or routine attention of specialists, requiring doctor's office visits, phone calls, etc.

### SUMMARY

A system for applying transcutaneous electrical stimulation can includes a wearable, such as a garment, sock, sleeve, brace, strap, etc. The wearable includes an electronic stimulator device that provides transcutaneous electrical stimulation to peripheral nerves for treatment of medical conditions. Advantageously, the wearable allows the subject to use the system at a time and place that is convenient. The subject may choose to use the device while they are at work or at home, or while walking, relaxing, or sleeping, as long as certain environments and/or activities (e.g., wet environments/activities) are avoided. Since there are no implantable or percutaneous components, the risk of infection, battery fault burns, and transcutaneous power transfer discomfort and/or bleeding, are greatly reduced or eliminated.

The wearable includes electrodes that are arranged in a predetermined pattern or array, and that engage the subject's skin at desired locations when the wearable is worn. These skin surface mounted electrodes can, for example, be similar to those of other transcutaneous electrical nerve stimulation (“TENS”) units to implement high voltage skin surface electrical stimulation. The electrodes include stimulating electrodes and recording electrodes, which the wearable can position at the same location or at different locations on the subject's skin. In fact, the identities of individual electrodes, i.e., stimulating or recording, can change depending on the application/treatment for which the system is being used. The stimulating electrodes apply the transcutaneous electrical stimulation to the subject's skin, and the recording electrodes record the electromyogram (EMG) responses elicited by the stimulation.

The wearable also includes a control unit that is electrically connected to the electrodes and that is operable to control electrical stimulation applied by the stimulating electrodes and to control the recording of EMG responses by the recording electrodes. The control unit can execute closed-loop control algorithms, which adjust stimulation patterns, periodically or constantly, based on the elicited EMG response from the recruited nerves as feedback. The control unit can execute open-loop control algorithms, which can administer stimulation patterns, periodically or constantly, with or without adjustment, and without feedback. The control unit can execute hybrid control algorithms, which execute both closed-loop and open-loop control algorithms based on stimulation and/or recording sensor indications.

Alternatively, instead of the EMG response providing the closed-loop feedback, or as a supplement to the EMG response, the system can include alternative devices, such as mechanomyogram (MMG) devices (e.g., an accelerometer), or can implement electronic measurements, such as electrode impedance, to implement the closed-loop control.

This closed-loop control can eliminate the need for “programming sessions” commonly required for neurostimulation systems. The day-to-day variability that arises due to electrode placement and skin impedance necessitates these sessions to make sure that the electrodes are positioned to provide adequate stimulation treatment. With the present system, instead of physically adjusting the electrode positions on the subject in order to find the arrangement that produces the desired response, the system itself can select which electrodes to use, and can adjust the number and pattern of electrodes until an acceptable response (EMG and/or MMG) is achieved. Once the appropriate electrodes pattern is identified, the order, intensity, timing, etc. of the stimulation can be further tuned or adjusted to optimize the EMG and/or MMG response. The system can tailor the electrical stimulation applied by each individually controllable electrode in the array so that the stimulation characteristics of each electrode (e.g., frequency, amplitude, pattern, duration, etc.) is configured to deliver the desired stimulation effect. This tailoring can be implemented automatically through the algorithm, which incrementally adjusts these characteristics, monitoring the and/or response at each increment until optimal settings are identified. Stimulation therapy can then be applied with these settings, according to the algorithm, which can be dictated by the requirements of the treating physician.

Throughout the electrical stimulation treatment process, the system can implement periodic or continuous measurement of system integrity. One such measurement is that of electrode impedance to remove the risks that can arise when electrodes lift away from the skin or certain properties of the electrodes deteriorate. The impedance measurement capability could also potentially be used to provide an indication of the optimal electrode location for nerve stimulation. This may be the case, for example, in areas where the skin is thin and where the stimulated nerves are most superficial. Thus, impedance values may be used as an input to the closed-loop stimulation algorithm to adjust stimulation patterns. By way of example, when stimulating the tibial nerve, the posterior area of the medial malleolus typically has comparatively thin skin and is the site where tibial nerve is most superficial, which leads to its being a good candidate for measuring electrode impedance.

The control unit and the architecture of the system may be designed to constantly optimize stimulation by monitoring the quality of nerve recruitment periodically or on a pulse-by-pulse basis, with the goal of keeping recruitment strength to a minimum (which can reduce muscle twitching) and to minimize the stimulation energy being delivered through the skin. The EMG recording feature is capable of detecting both M-wave and F-wave responses, which can be used as feedback inputs (together or independently) to the closed-loop stimulation algorithm to determine the level of activation of the stimulated peripheral nerve. A significant aspect of the F-wave is that it provides an indication that the stimulation-evoked peripheral nerve action potential has activated motor neurons in the associated spinal cord nerves/nerve plexus. For example, an F-wave response to tibial nerve stimulation indicates that the tibial nerve action potential has activated motor neurons in the sacral spinal cord/sacral plexus.

The wearable transcutaneous electrical stimulation device can be used to stimulate various peripheral nerves in order to treat medical conditions associated with those nerves. For example, the system can be used to apply electrical stimulation to the tibial nerve to treat pelvic floor dysfunction, e.g., overactive bladder (OAB) medical conditions. As another example, the system can be used to apply electrical stimulation to the tibial nerve to treat sexual dysfunction. In this manner, it is believed that tibial nerve stimulation could be used to treat genital arousal aspects of female sexual interest/arousal disorder by improving pelvic blood flow. In yet another example, the system can be used to apply electrical stimulation to the tibial nerve to treat plantar fasciitis.

As another example, the system can be applied to the wrist area to provide stimulation to the ulnar nerve and/or median nerve. The stimulation electrode array can, for example, be placed on the inside of the lower arm anywhere 0 to 20 cm from the wrist line. EMG recording electrodes can be placed on the base of thumb to record signal from abductor/flexor pollicis brevis. EMG recording electrodes alternatively or additionally can be placed on the base of pinky to record signal from abductor/flexor digiti minimi brevis. The nerve activation could be confirmed by recording M-wave and F-wave EMG signals from the relevant muscles. The EMG signal can also be used as a control signal to adjust the stimulation parameters or stimulation electrode patterns. This technology can be applied to median nerve activation for pain management in carpal tunnel syndrome, hypertension management, and nerve conduction study/nerve injury diagnosis for median/ulnar nerve neuropathy, etc.

As a further example, the system can be used to apply transcutaneous electrical stimulation to provide neurostimulation to peripheral nerves in order to enhance nerve regeneration after peripheral nerve injury.

Implementing closed-loop control, the system can utilize measured EMG responses to detect and obtain data related to the electrical activity of muscles in response to the applied stimulation. This data can be used as feedback to tailor the application of the electrical stimulation. Additionally or alternatively, the system can also implement MMG sensors, such as accelerometers, to measure the physical response of the muscles. Other feedback, such as impedance measurements between electrodes and other biopotential recording, can also be utilized. Through this closed-loop implementation, the system can utilize techniques such as current steering and nerve localization to provide peripheral nerve stimulation therapy for treating various medical conditions.

The system, method, and apparatus for applying transcutaneous electrical stimulation disclosed herein has many aspects, which can be included or utilized in various combinations.

According to one aspect, a system, method, and apparatus treats a condition by applying transcutaneous electrical stimulation to a target peripheral nerve of a subject.

According to another aspect, alone or in combination with any other aspect, the system, method, and apparatus can include positioning a plurality of stimulation electrodes on a skin surface proximate the targeted peripheral nerve. The method also can include positioning one or more recording electrodes on a skin surface remote from the stimulation electrodes at a location where electromyogram (EMG) responses to electrical stimulation of the targeted peripheral nerve can be detected. The method also can include stimulating the peripheral nerve by applying electrical stimulation pulses via the stimulation electrodes according to stimulation parameters under closed-loop control in which EMG responses to the electrical stimulation pulses are monitored via the recording electrodes and the stimulation parameters are adjusted in response to the monitored EMG responses.

According to another aspect, alone or in combination with any other aspect, the system, method, and apparatus can include, in response to detecting an unacceptable condition of the recording electrodes, applying electrical stimulation pulses via the stimulation electrode pattern according to the stimulation parameters under open-loop control in which the stimulation parameters are maintained without adjustment.

According to another aspect, alone or in combination with any other aspect, the system, method, and apparatus can include a plurality of electrical stimulation electrodes spaced from each other in a predetermined configuration, one or more recording electrodes, a structure for supporting the stimulation electrodes and the recording electrodes spaced apart from each other, and a control unit for controlling the operation of the stimulation electrodes and the recording electrodes. The control unit is configured to energize the stimulation electrodes under closed-loop control using the recording electrodes to measure feedback, energize the stimulation electrodes under open-loop without measuring feedback, and determine whether to energize the stimulation electrodes under closed-loop control or open-loop control based on determining whether the feedback measured by the recording electrodes is reliable.

According to another aspect, alone or in combination with any other aspect, the structure for supporting the stimulation electrodes and the recording electrodes can include a wearable structure configured to position the stimulation electrodes in the proximity of a peripheral nerve and to position the recording electrodes in the proximity of a muscle activated by the peripheral nerve.

According to another aspect, alone or in combination with any other aspect, the wearable structure can position the stimulation electrodes proximate the peripheral nerve and the recording electrodes proximate a location where EMG signals that result from recruitment of the peripheral nerve's motor fibers can be detected.

According to another aspect, alone or in combination with any other aspect, the wearable structure can include a strap, wherein the stimulation electrodes and recording electrodes are positioned at different locations along the length of the strap. The strap can be configured to have a portion wrapped around the subject's ankle to position the stimulating electrodes proximate the tibial nerve between the medial malleolus and the Achilles tendon. The strap can also be configured to have a portion wrapped around the subject's foot to position the recording electrodes on the bottom of the subject's foot near the abductor hallucis and the flexor hallucis brevis.

According to another aspect, alone or in combination with any other aspect, the wearable structure can include a brace comprising an upper portion upon which the stimulation electrodes are positioned and a lower portion upon which the recording electrodes are positioned. The upper portion of the brace can be configured to be wrapped around the subject's ankle to position the stimulating electrodes proximate the tibial nerve between the medial malleolus and the Achilles tendon. The lower portion of the brace can be configured to be wrapped around the subject's foot to position the recording electrodes on the bottom of the subject's foot near the abductor hallucis and the flexor hallucis brevis.

According to another aspect, alone or in combination with any other aspect, the phase relationship or time delay can be indicative of the foot, right or left, upon which the apparatus is worn.

According to another aspect, alone or in combination with any other aspect, the wearable garment can include an ankle brace and the stimulating electrodes can include left-side stimulating electrodes and right-side stimulating electrodes configured so that the left-side electrodes are positioned adjacent the tibial nerve near the medial malleolus when worn on the right foot, and so that the right-side electrodes are positioned adjacent the tibial nerve near the medial malleolus when worn on the left foot.

According to another aspect, alone or in combination with any other aspect, the control unit can be configured to select whether to use the left-side electrodes or right-side electrodes in response to determining the foot upon which the apparatus is worn.

According to another aspect, alone or in combination with any other aspect, the apparatus can include a plurality of stimulation electrodes, and the control unit can be configured to select which of the stimulation electrodes to utilize. The control unit can also be configured to select stimulation electrode pairs and measure the impedance between the selected pairs. The control unit can be further configured to determine the foot upon which the apparatus is worn in response to the measured impedance.

According to another aspect, an apparatus for applying electrical stimulation to a target peripheral nerve in a subject includes a wearable structure configured to be worn on at least one of the subject's lower leg, foot, or ankle. A first stimulation electrode is mounted on the wearable structure at a first location on the wearable structure. A second stimulation electrode is mounted on the wearable structure at a second location on the wearable structure, different than the first location on the wearable structure. One or more recording electrodes are mounted on the wearable structure. The apparatus also includes a control unit for controlling the operation of the first stimulation electrode, the second stimulation electrode, and the one or more recording electrodes. The wearable is configured to position the first stimulation electrode on a medial side of the target peripheral nerve, and to position the second stimulation electrode on a lateral side of the target peripheral nerve. The control unit is also configured to use the first and second stimulation electrodes to apply electrical stimulation to the target peripheral nerve and to record physiological responses to the applied electrical stimulation using the recording electrodes.

According to another aspect, alone or in combination with any other aspect, the controller can be configured to apply the electrical stimulation to the target peripheral nerve using one of the first and second stimulation electrodes as a cathode and the other of the first and second stimulation electrodes as an anode.

According to another aspect, alone or in combination with any other aspect, the controller can be configured to apply cathodic electrical stimulation to the target peripheral nerve.

According to another aspect, alone or in combination with any other aspect, the controller can be configured to apply anodic electrical stimulation to the target peripheral nerve.

According to another aspect, alone or in combination with any other aspect, the controller can be configured to switch between applying cathodic and anodic electrical stimulation to the target peripheral nerve.

According to another aspect, alone or in combination with any other aspect, the target peripheral nerve can be the tibial nerve. The wearable can be configured to position the first stimulation electrode medially of the tibial nerve, and to position the second stimulation electrode laterally of the tibial nerve.

According to another aspect, alone or in combination with any other aspect, the controller can be configured to apply electrical stimulation laterally across the tibial nerve in a direction that acts perpendicular to the axon fibers in the tibial nerve.

According to another aspect, alone or in combination with any other aspect, the wearable can be configured to position the first stimulation electrode on a skin surface of the subject located on a medial side of a cavity between an Achilles tendon, tibia bone, and fibula bone of the subject through which the tibial nerve extends, and to position the second stimulation electrode on a skin surface of the subject located on a lateral side of the cavity.

According to another aspect, alone or in combination with any other aspect, the controller can be configured to apply electrical stimulation laterally across the cavity from the medial side to the lateral side and vice versa.

According to another aspect, alone or in combination with any other aspect, the apparatus can also include electrical traces secured to the wearable structure. The electrical traces can be configured to electrically connect the stimulation electrodes and recording electrodes to the control unit.

According to another aspect, alone or in combination with any other aspect, the control unit can be configured to detect via the recording electrodes the presence of an EMG response to stimulation therapy. The control unit can be configured to, in response to detecting no EMG response, deliver stimulation therapy under open-loop control without EMG feedback. The control unit can also be configured to, in response to detecting an EMG response, deliver stimulation therapy under closed-loop control with EMG feedback.

According to another aspect, alone or in combination with any other aspect, the control unit can be configured to detect via the stimulating electrodes whether the apparatus is being worn on a right foot or left foot of the user. In response to detecting the foot, the control unit can also be configured to determine which stimulation electrode is configured as a cathode and which stimulation electrode is configured as an anode.

According to another aspect, alone or in combination with any other aspect, the wearable can include an ankle brace having a first portion configured to be strapped around a foot and to position the one or more recording electrodes on the foot. The ankle brace can also include a second portion configured to be strapped around an ankle to position the first stimulation electrode on a medial side of the target peripheral nerve, and to position the second stimulation electrode on a lateral side of the target peripheral nerve.

According to another aspect, alone or in combination with any other aspect, the wearable can include a strap having a first portion configured to be wrapped around a foot and to position the one or more recording electrodes on the foot. The strap can also include a second portion configured to be strapped around an ankle to position the first stimulation electrode on a medial side of the target peripheral nerve, and to position the second stimulation electrode on a lateral side of the target peripheral nerve.

### DRAWINGS

FIG. 1A illustrates a left-foot implementation of an electronic medical device for delivering transcutaneous electrical stimulation of peripheral nerves, according to a first example configuration.

FIG. 1B illustrates a right-foot implementation of the electronic medical device for delivering transcutaneous electrical stimulation of peripheral nerves, according to the first example configuration.

FIG. 2A is an inner surface plan view of the electronic medical device of FIGS. 1A and 1B.

FIG. 2B is an outer surface plan view of the electronic medical device of FIGS. 1A and 1B.

FIGS. 2C-E are outer surface plan views of the electronic medical device of FIGS. 1A and 1B illustrating sequential steps in preparing the device for use.

FIG. 3A illustrates a left-foot implementation of an electronic medical device for delivering transcutaneous electrical stimulation of peripheral nerves, according to a second example configuration.

FIG. 3B illustrates a right-foot implementation of the electronic medical device for delivering transcutaneous electrical stimulation of peripheral nerves, according to the second example configuration.

FIG. 4A is an inner surface plan view of components of the electronic medical device of FIGS. 3A and 3B.

FIG. 4B is an outer surface plan view of the components of the electronic medical device of FIGS. 3A and 3B.

FIG. 4C is an outer surface plan view, taken from a first side, illustrating the components of FIGS. 4A and 4B assembled to form the electronic medical device of FIGS. 3A and 3B.

FIG. 4D is an outer surface plan view, taken from a second side, opposite the first side, illustrating the components of FIGS. 4A and 4B assembled to form the electronic medical device of FIGS. 3A and 3B.

FIG. 5 is a schematic block diagram of a control unit portion of the electronic medical device.

FIG. 6 is a diagram illustrating example electrode arrangements for portions of the electronic medical device.

FIG. 7 is a flow chart illustrating an example nerve localization process implemented by the electronic medical device.

FIG. 8 is a series of charts illustrating examples of recorded EMG responses to electrical nerve stimulation.

FIG. 9 is a flow chart illustrating an example open-loop and closed-loop electrical nerve stimulation processes implemented by the electronic medical device.

FIG. 10 illustrates the anatomy of a human foot.

FIG. 11 illustrates an electronic medical device for delivering transcutaneous electrical stimulation of peripheral nerves, according to another example configuration.

FIG. 12 illustrates an electronic medical device for delivering transcutaneous electrical stimulation of peripheral nerves, according to another example configuration.

FIG. 13 illustrates recording electrode placements for the electronic medical devices of FIGS. 11 and 12.

FIG. 14 is a graph that illustrates the effect of the size of recording electrodes of the electronic medical device.

FIG. 15 is a graph that illustrates the effect of switching the electronic medical device between the feet of a subject.

FIG. 16 is a graph that illustrates a method for determining optimal charge for neurostimulation.

FIG. 17 is a graph that illustrates adjusting the optimal charge in response to adjusting the applied current amplitude for neurostimulation.

FIG. 18 is a graph that illustrated an operating zone within which neurostimulation can be executed.

FIGS. 19A-19C are examples of interpolated target therapy ranges.

FIGS. 20 and 21 are flow chards that illustrate two different methods for determining target stimulation.

FIG. 22 is a flow chart that illustrates a method by which to control the application of stimulation therapy.

FIG. 23 is a flow chart that illustrates another method by which to control the application of stimulation therapy.

FIG. 24 is a perspective view of the medial side of a right lower leg, ankle, and foot illustrating the anatomy thereof.

FIG. 25 is a posterior view of lower legs, ankles, and feet illustrating an example electrode placement for bilateral stimulation.

FIG. 26 illustrates an electronic medical device for delivering transcutaneous electrical stimulation of peripheral nerves, according to another example configuration.

FIG. 27A is a chart illustrating cathode stimulation.

FIG. 27B is a chart illustrating anode stimulation.

FIG. 28 illustrates an electronic medical device for delivering transcutaneous electrical stimulation of peripheral nerves, according to another example configuration.

FIG. 29 illustrates an electronic medical device for delivering transcutaneous electrical stimulation of peripheral nerves, according to another example configuration.

### DESCRIPTION

An electronic medical device, a system including the medical device, and a method for using the medical device, is configured to apply transcutaneous electrical stimulation to peripheral nerves to treat various medical conditions.

For example, the system can be used to stimulate the tibial nerve (transcutaneous tibial nerve stimulation “TTNS”) to treat medical conditions associated with pelvic floor dysfunction, e.g., over-active bladder (OAB). In a TTNS implementation, the electronic medical device applies electrical stimulation near the medial malleolus, which activates both sensory and motor fibers in the nerve. The activation of the sensory fibers of the tibial nerve helps to treat the urge-related symptoms of OAB. The activation of the motor fibers can, however, cause unwanted side effects, such as toe twitch or spasm.

As another example, the system can be used to apply electrical stimulation to the tibial nerve to treat sexual dysfunction. In this manner, it is believed that tibial nerve stimulation could be used to treat genital arousal aspects of female sexual interest/arousal disorder by improving pelvic blood flow.

As another example, the system can be applied to the wrist area to provide stimulation to the ulnar nerve and/or median nerve for pain management in carpal tunnel syndrome, hypertension management, and nerve conduction study/nerve injury diagnosis for median/ulnar nerve neuropathy, etc.

The system and/or the device employed by the system can have a variety of implementations. According to one implementation, the electronic medical device (i.e., the electrodes, control unit, wiring, etc.) can be fixed to a garment that is worn by the subject. The garment can be tight or snug-fitting so as to maintain sufficient contact between the subject's skin and can be configured to position the electrodes at locations specific to the peripheral nerves being stimulated. For example, to stimulate peripheral nerves in the area of the foot or ankle, such as the tibial nerve near the medial malleolus as described above, the garment can be in the form of a sock, ankle brace, strap, sleeve, or other like structure. For stimulating peripheral nerves on the leg, the garment can be a brace, strap, or sleeve sized appropriately for lower leg, knee, or upper leg positioning. For knee or ankle positioning, the garment can be configured, e.g., with openings, slots, or interconnected sections, to allow for bending with the joint while maintaining electrode positioning and contact.

Similarly, for stimulating peripheral nerves on the hand, the garment can be in the form of a glove, mitten, hand brace, or sleeve. For stimulating peripheral nerves on the arm, the garment can be a tight/snug fitting brace, strap, or sleeve (e.g., neoprene) that is sized appropriately for lower arm (forearm/wrist), elbow, or upper arm positioning. For wrist and/or elbow positioning, the sleeve can be configured, e.g., via openings, slots, or interconnected sections, to allow for bending with the joint while maintaining electrode positioning and contact.

In keeping with the above, it will be appreciated that the manner in which the electronic medical device can be secured or supported on the subject can vary. It will also be appreciated that the manner in which the electronic medical device is supported is not critical, as long as contact between the electrodes and the subject's skin is maintained, the positions of the electrode on the subject are maintained, and that the aforementioned are achieved in a manner that is comfortable to the subject.

Strap Implementation

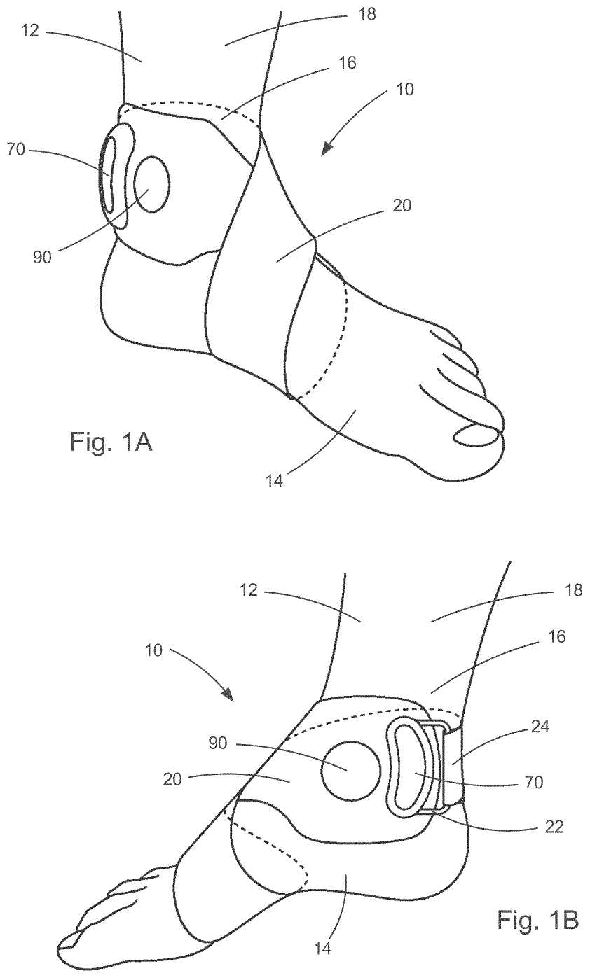

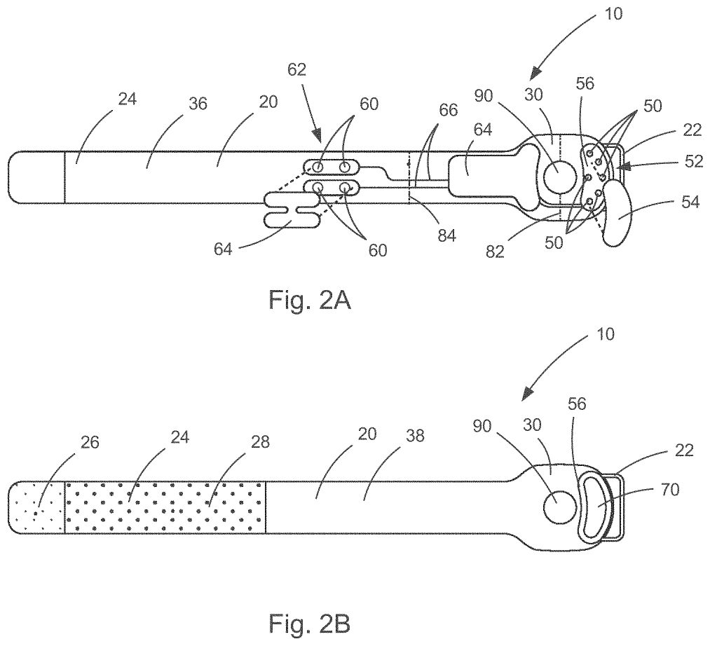

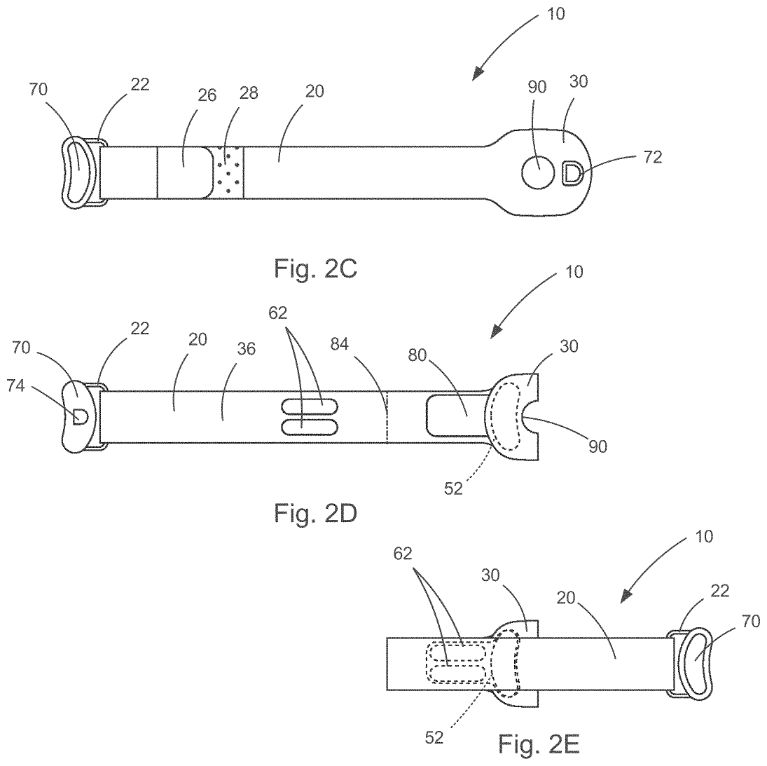

FIGS. 1A-B illustrate a system comprising an example configuration of the electronic medical device 10 for providing transcutaneous electrical nerve stimulation, referred to herein as a neurostimulator, supported on a subject 12. The neurostimulator 10 of FIGS. 1A-B includes a garment in the form of a strap 20 that supports the neurostimulator and its components on the subject 12. In the example configuration of FIGS. 1A-B, the strap 20 connects the neurostimulator 10 to the subjects foot 14, with FIG. 1A illustrating a left foot implementation, and FIG. 1B illustrating a right foot implementation. In both instances, the strap 20 is wrapped figure-eight style, with one loop extending around the foot and one loop extending around the lower leg/ankle. Opposite end portions of the strap 20 can be interconnected, e.g., via a buckle or loop 22 and an end portion 24 of the strap that extends through the loop, is folded over, and connected to itself with a hook and loop fastener. The hook and loop fastener is shown in FIG. 2B and includes a hook portion 26 and loop portion 28.

The strap 20 implementation of the neurostimulator 10 is advantageous in that it is versatile and can be adapted to secure the neurostimulator to a wide variety of locations on the subject 12. The strap 20 can easily be wrapped around the foot 14 and/or ankle 16, as shown, and can also be wrapped around and secured to any location along the length of the subject's leg 18, either in a single loop or more than one loop, as the length of the strap permits. At the knee, the strap 20 can be wrapped, for example, in a figure-eight style in a manner similar to that illustrated in FIGS. 1A and 1B.

Referring to FIGS. 2A-B, the neurostimulator 10 includes a several of components that are secured or otherwise supported on the strap 20. The securement of these components can be achieved in a variety of manners, such as by adhesives, stitching, mechanical fastening, hook and loop fasteners, or a combination thereof.

The neurostimulator 10 includes stimulation electrodes 50 that are arranged in one or more arrays 52 and positioned on an inner surface 36 of the strap 20 at a widened end portion 30 of the strap. The number of stimulation electrodes 50, the area covered by the array 52, the electrode density (i.e., number of electrodes per unit area) in the array, and the distribution or pattern of electrodes within the array all can vary depending on the intended application of the neurostimulator 10. Additionally, the neurostimulator 10 can include more than one stimulation electrode array 52 again, depending on the application. In the example configuration of FIG. 2A, the stimulation electrode array 52 includes six stimulation electrodes 50 arranged in a generally elongated kidney-shaped manner. The number and arrangement of the stimulation electrodes 50, and the location/position of the electrode array 52 on the strap 20 are by way of example only and are by no means limiting.

In the example configuration of FIG. 2A, the stimulation electrodes 50 can be dry electrodes, in which case the neurostimulator 10 can include a removable/replaceable stimulation gel pad 54 shaped and sized to coincide with and cover the stimulation electrode array 52. In use, the gel pad 54 facilitates a strong, reliable electrical connection between the stimulation electrodes 50 and the subject's skin.

The neurostimulator 10 also includes dedicated recording electrodes 60 that are arranged in one or more arrays 62 and positioned on the inner surface 36 of the strap 20 spaced from the stimulation electrode array 52. The spacing between the stimulation electrodes 50 and the recording electrodes 60 can be important, as it can be necessary to provide adequate distance between the electrodes so that electrical stimulation signals can be separated or distinguished from responses (e.g., neurological, muscular, neuromuscular, etc.) to those electrical stimulation signals. This facilitates utilizing responses to stimulation sensed by the recording electrodes 60 as feedback in a closed-loop stimulation control scheme, which is described in detail below.

The number of recording electrodes 60, the area covered by the array 62, the electrode density (i.e., number of electrodes per unit area) in the array, and the distribution or pattern of electrodes within the array all can vary depending on the intended application of the neurostimulator 10. Additionally, the neurostimulator 10 can include more than one recording electrode array 62 again, depending on the application. In the example configuration of FIG. 2A, the recording electrode array 62 includes four electrodes 60 arranged linearly in two parallel rows of two electrodes. The number and arrangement of the recording electrodes 60, and the location/position of the electrode array 62 on the strap 20 are by way of example only and are by no means limiting.

In the example configuration of FIG. 2A, like the stimulation electrodes 50, the recording electrodes 60 can also be dry electrodes. Because of this, the neurostimulator 10 can also include a removable/replaceable gel pad 64 shaped and sized to coincide with and cover the recording electrode array 62. In use, the gel pad 54 facilitates a strong, reliable electrical connection between the recording electrodes 60 and the subject's skin.

Referring to FIG. 2B, the neurostimulator 10 also includes an electronic control unit 70 that is operative to control the application of transcutaneous electrical nerve stimulation via the stimulating electrodes 50 and to receive stimulation feedback gathered by the recording electrodes 60. The control unit 70 is located at the widened end 30 of the strap 20 on an outer surface 38, opposite the inner surface 36, of the strap 20. The buckle 22 can be a portion of the control unit 70 or can be connected to the control unit. In the example configuration of FIG. 2B, the control unit 70 has a generally elongated kidney-shaped configuration similar to that of the stimulating electrode array 52 and is positioned on the outer surface 38 generally opposite the stimulating electrode array. This is by no means necessary to the design of the neurostimulator 10, as the shape and location of the control unit 70 can vary.

In the example configuration of FIG. 2B, however, the shape and the positioning of the control unit 70 is convenient. The control unit 70 is detachably connected to the remainder of the neurostimulator 10 via a plug-in or snap-in connector 72 (see FIG. 2B), which receives a mating connector 74 (see FIG. 2D) on the control unit 70. FIG. 2B shows the control unit 70 connected to the neurostimulator 20 via the connector 72, and FIG. 2C shows the neurostimulator 20 with the control unit detached from the connector and removed. Configuring the control unit 70 to be detachable/removable allows the control unit to be utilized with other neurostimulator configurations and also allows the strap 20 and the components remaining on the strap (e.g., the electrodes, etc.) to be replaced when worn out, expired, or otherwise due for replacement.

Advantageously, the stimulating electrode array 52 can be part of an assembly in which the stimulating electrodes 50 can be mounted on a substrate or housing 56 constructed, for example of plastic. This substrate/housing 56 can itself be secured to the strap 20 (e.g., via adhesives, stitching, or mechanical fastening) to thereby secure the stimulation electrode array 52 to be strap. Forming the stimulating electrode array 52 in this manner facilitates a precise arrangement and spacing of the stimulation electrodes 50 and makes it easy to secure them to the strap 20.

The connector 72 can also be formed as a portion of the housing 56. The connector 72 can be configured to protrude from a side of the housing 56 opposite the stimulation electrodes 50. The connector 72 can, for example, extend through a hole in the strap 20 to position the connector on or extending from the outer surface 38. When the control unit 70 is connected to the connector 72, the strap 20 can be positioned between the control unit and the portion of the housing 56 supporting the stimulator electrode array 52.

The connector 72 can support a plurality of terminals for electrically connecting the control unit 70 to the stimulation electrodes 50 and the recording electrodes 60. Certain terminals in the connector 72 can be electrically connected to the stimulation electrodes 50 by wires or leads that are embedded within the plastic housing material (e.g., via insert molding). Embedding the leads in this manner helps maintain adequate spacing between the conductors, which avoids the potential for shorts in the circuitry.

Other terminals in the connector can be electrically connected to the recording electrodes 60 by wires or leads 66 that are partially embedded within the plastic housing material (e.g., via insert molding) and pass through the housing 56, extending to the feedback electrode arrays 62. Through this configuration, all of the necessary electrical connections to the stimulation and recording electrodes 50, 60 are made when the control unit 70 is installed on the connector 72.

The neurostimulator 10 also includes electrode backing 80 that facilitates safe storage and portability of the system. Fold lines 82, 84 shown in FIG. 2A indicate lines along which the neurostimulator 10/strap 20 can be folded to place the device in the stored condition. The steps involved in placing the neurostimulator 10 in the stored condition are illustrated in FIGS. 2C-2E.

As shown in FIG. 2C, the control unit 70 is detached from the housing 56. The control unit 70 is secured to the end portion 24 of the strap 20 by the hook and loop fastener 26, 28. Next, as shown in FIG. 2D, with the inner surface 36 facing up, the widened end portion 38 is folded over along the fold line 82, which places the stimulating electrode array 52 on a corresponding portion of the electrode backing 80. Next, as shown in FIG. 2E, the strap 20 is folded over along the fold line 84, which places the recording electrode array 62 on a corresponding portion of the electrode backing 80. This leaves the neurostimulator 10 in the stored condition of FIG. 2E.

To use the neurostimulator 10, the strap 20 is simply unfolded and the control unit 70 is connected to the housing 56 via their respective connectors 72, 74. The hook and loop fastener 26, 28 can be disconnected, the strap 20 wrapped around the appropriate anatomy of the subject, and the fastener re-connected to attach neurostimulator 10 to the subject. Conveniently, where the neurostimulator 10 is configured for stimulating the tibial nerve in the position illustrated in FIGS. 1A-B, the widened end 30 of the strap 20 can include a visual alignment cue 90, such as a hole in the strap, that becomes aligned with the medial malleolus of the ankle when the stimulating electrodes are properly positioned.

Brace Implementation

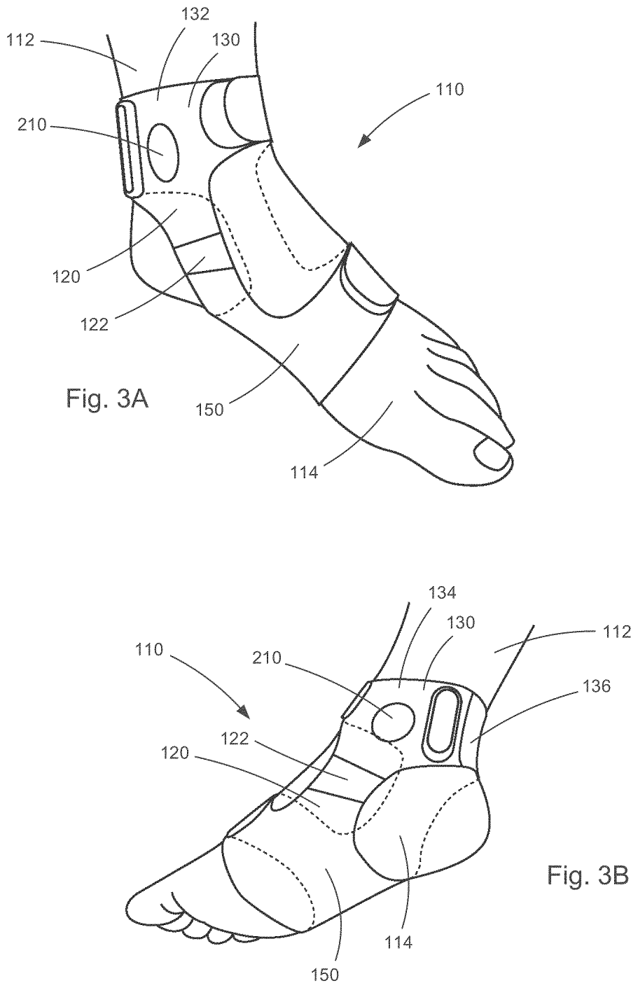

FIGS. 3A-B illustrate a system comprising another example configuration of an electronic medical device 110 for providing transcutaneous electrical nerve stimulation, referred to herein as a neurostimulator, supported on a subject 112. The neurostimulator 110 of FIGS. 3A-1B includes a garment in the form of a brace 120 that supports the neurostimulator and its components on the subject 112. In the example configuration of FIGS. 3A-B, the brace 120 connects the neurostimulator 110 to the subject's foot 114, with FIG. 3A illustrating a left foot implementation, and FIG. 3B illustrating a right foot implementation. In both instances, the brace 120 has an upper portion 130 wrapped around the lower leg/ankle and a lower portion 150 portion wrapped around the foot/ankle. Each of these portions are secured to the subject via a connection such as a hook and loop fastener.

The brace 120 implementation of the neurostimulator 10 is advantageous in that it is versatile in its ability to position the stimulating electrodes and recording electrodes at different locations on the subject. For example, stimulating electrodes can be positioned on the upper portion 130 of the brace 120 wrapped around the ankle, and recording electrodes can be positioned on the lower portion 150 of the brace wrapped around the foot. This can be especially advantageous for closed-loop neurostimulation of the tibial nerve. In this implementation, stimulating electrodes on the upper portion 130 can be located between the medial malleolus and the Achilles tendon to provide electrical stimulation to the tibial nerve. Recording electrodes on the lower portion 150 can be located on the bottom of the subject's foot, near the flexor muscles (abductor hallucis and the flexor hallucis brevis) for the big toe and can record the EMG signals that result from recruitment of the tibial nerve's motor fibers.

As another advantage, the brace 120 is configured for placement at or about a subject's joint and provides for movement of that joint. While the brace 120 is illustrated as being applied at the subject's ankle joint, it will be appreciated that the brace 120 can also be applied at the knee joint or elbow joint. Additionally, positioning the brace 120 at a joint is not critical, as it can be seen that the brace can be applied at any location along the subject's arms or legs, size permitting.

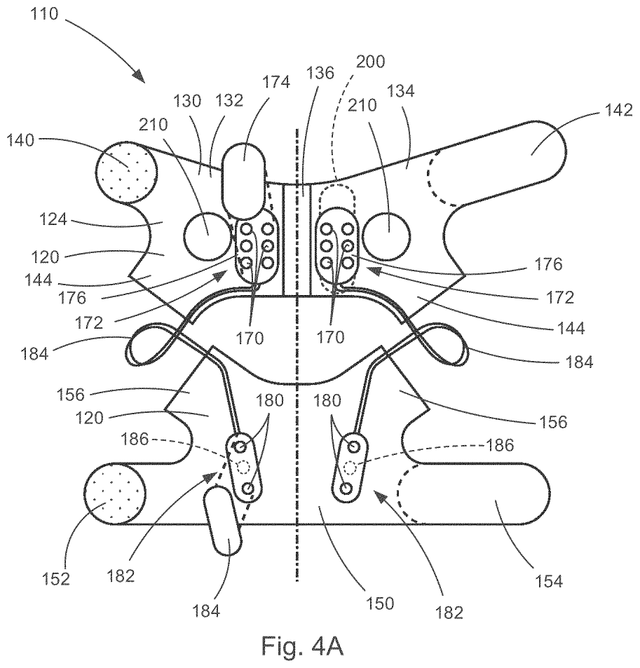

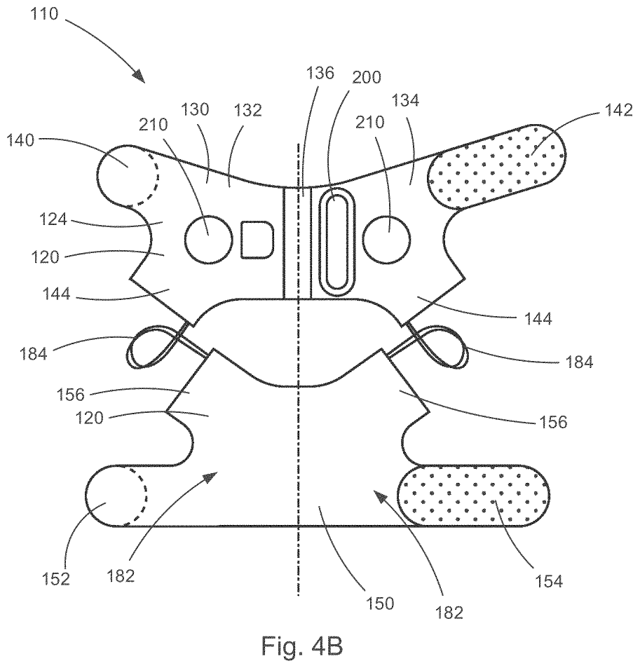

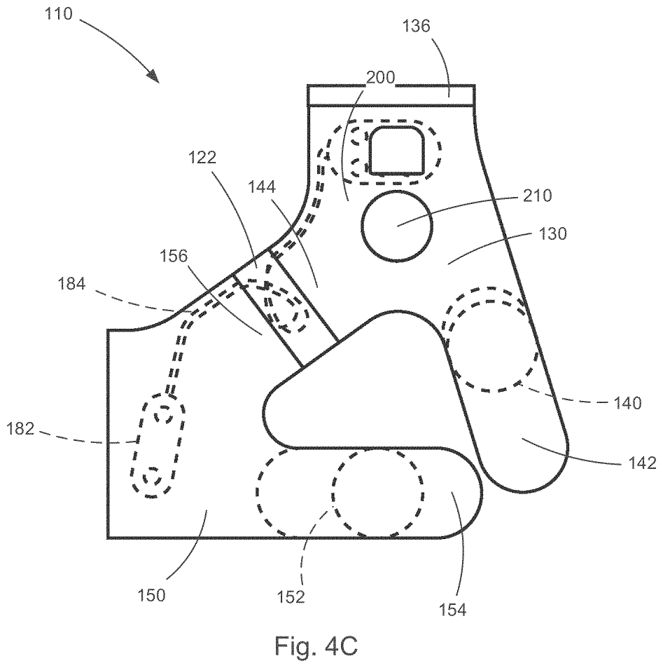

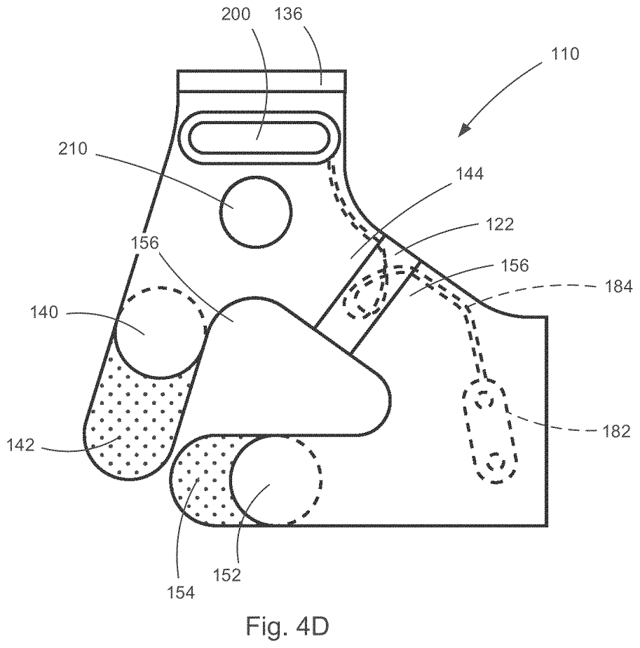

The construction of the neurostimulator 110 is illustrated in FIGS. 4A-D. For the example configuration of FIGS. 4A-D the upper portion 130 and lower portion 150 of the strap 120 are separate components that are interconnected by adjustment bands 122. The adjustment bands 122 can allow for adjusting the spacing between the upper and lower portions 130, 150, e.g., via a buckle or hook and loop fastener, or the bands can be of a fixed size amongst a range of sizes, e.g., x-small, small, medium, large, x-large, etc. The respective sizes of the upper and lower portions 130, 150 can be similarly sized. In fact, the upper portion 130 can itself be composed of first and second portions 132, 134 connected by a band 136 that allows for adjusting the spacing between the upper and lower portions 130, 150, e.g., via a buckle or hook and loop fastener.

The upper portion 130 of the brace 120 includes a hook and loop fastener composed of a hook portion 140 and a loop portion 142, which are positioned opposite each other along an upper extent of the upper portion. The upper portion 130 also includes opposite tab portions 144 to which the adjustment tabs 122 (see, FIGS. 4C-D) are connected, e.g., via stitching. Similarly, the lower portion 130 of the brace includes a hook and loop fastener composed of a hook portion 152 and a loop portion 154, which are positioned opposite each other along a lower extent of the lower portion. The lower portion 150 also includes opposite tab portions 156 to which the adjustment tabs 122 (see, FIGS. 4C-D) are connected, e.g., via stitching.

The neurostimulator 110 includes a several of components that are secured or otherwise supported on the brace 120. The securement of these components can be achieved in a variety of manners, such as by adhesives, stitching, mechanical fastening, hook and loop fasteners, or a combination thereof. FIGS. 4A and 4B illustrate the neurostimulator 110 in a partially assembled condition, with the electronic components of the neurostimulator mounted on the brace 120 prior to the first and second portions 132, 134 being interconnected by the adjustment bands 122. This construction is advantageous because it allows the electronic components of the neurostimulator 110 to be assembled onto brace 120 while the upper and lower portions 130, 150 lie flat. The lying flat illustration of FIGS. 4A-B is for purposes of simplicity as it allows the upper and lower portions 130, 150 to be illustrated lying flat. FIG. 4A illustrates an inner surface 124 of the brace 120. FIG. 4B illustrates an outer surface 126 of the brace 120.

The neurostimulator 110 includes stimulation electrodes 170 that are arranged in one or more arrays 172 and positioned on the inner surface 124 of the upper portion 130 of the brace 120. In the example configuration illustrated in FIG. 4A, the stimulation electrode arrays 172 are positioned on opposite sides of the adjustment band 136 connecting the first and second portions 132, 134 of the upper portion 130. This arrangement can, for example, allow the brace 130 implementation of the neurostimulator 110 to be ambidextrous.

The number of stimulation electrodes 170, the area covered by the stimulation electrode arrays 172, the electrode density (i.e., number of electrodes per unit area) in the arrays, and the distribution or pattern of electrodes within the array all can vary depending on the intended application of the neurostimulator 110. In the example configuration of FIG. 4A, each stimulation electrode array 172 includes six stimulation electrodes 170 arranged in a generally rectangular manner in two rows of three electrodes. The number and arrangement of the stimulation electrodes 170, and the location/position of the electrode array 172 on the brace 120 are by way of example only and are by no means limiting.

In the example configuration of FIG. 4A, the stimulation electrodes 170 can be dry electrodes, in which case the neurostimulator 110 can include one or more removable/replaceable stimulation gel pads 174 shaped and sized to coincide with and cover the stimulation electrode array 172. In use, the gel pads 174 facilitate a strong, reliable electrical connection between the stimulation electrodes 170 and the subject's skin.

The neurostimulator 110 also includes recording electrodes 180 that are arranged in one or more arrays 182 and positioned on the inner surface 124 of the lower portion 150 of the brace 120 at a location spaced from the stimulation electrode arrays 172. The spacing between the stimulation electrodes 170 and the recording electrodes 180 can be important, as it can be necessary to provide adequate distance between the electrodes so that electrical stimulation signals can be separated or distinguished from responses (e.g., neurological, muscular, neuromuscular, etc.) to those electrical stimulation signals. This facilitates utilizing responses to stimulation sensed by the recording electrodes 180 as feedback in a closed-loop stimulation control scheme which, again, is described in detail below.

The number of recording electrodes 180, the area covered by the array 182, the electrode density (i.e., number of electrodes per unit area) in the array, and the distribution or pattern of electrodes within the array all can vary depending on the intended application of the neurostimulator 110. In the example configuration of FIG. 4A, there are two recording electrode arrays 182, each of which includes two recording electrodes 180 arranged linearly. The number and arrangement of the recording electrodes 180, and the location/position of the electrode arrays 182 on the brace 120 are by way of example only and are by no means limiting.

In another implementation, the neurostimulator 110 can be configured to include MMG sensors (e.g., accelerometers) for sensing muscle movement as opposed to electrical activity. The optional MMG sensors are illustrated in dashed lines at 186 in FIG. 4A. In this implementation, the MMG sensors 186 can be implemented in addition to or in place of, the EMG electrodes 180. Implementing the MMG 186 sensors along with the EMG sensors 180 can prove beneficial in that the combination can provide additional functionality. For example, the MMG sensor 186 can be used to confirm the validity of an EMG measured feedback response. Additionally, the MMG sensors 186 (or any other accelerometer for that matter) can be used to verify that the subject in a resting, i.e., not moving, condition prior to initiating a therapy session.

In the example configuration of FIG. 4A, like the stimulation electrodes 170, the recording electrodes 180 can also be dry electrodes. Because of this, the neurostimulator 110 can also include a removable/replaceable recording gel pad 184 shaped and sized to coincide with and cover the recording electrode arrays 182. In use, the gel pad 184 facilitates a strong, reliable electrical connection between the recording electrodes 180 and the subject's skin.

Referring to FIG. 4B, the neurostimulator 110 also includes an electronic control unit 200 that is operative to control the application of transcutaneous electrical nerve stimulation via the stimulating electrodes 170 and to receive stimulation feedback gathered by the recording electrodes 180. The control unit 200 is located on the outer surface 126 of the upper portion 130 adjacent the adjustment band 136 and opposite one of the stimulating electrode arrays 172 on the inner surface 124 of the upper portion. In the example configuration of FIG. 4B, the control unit 200 has a generally elongated racetrack-shaped configuration similar, to that of the stimulating electrode arrays 172, although narrower. This is by no means necessary to the design of the neurostimulator 110, as the shape and location of the control unit 200 can vary.

In the example configuration of FIG. 4B, however, the shape and the positioning of the control unit 200 is convenient. The control unit 200 can be detachably connected to the remainder of the neurostimulator 110 via a plug-in or snap-in connector, such as by a connector (not shown) that is similar or identical to the connector associated with the control unit of the example configuration of FIGS. 2A-D. Configuring the control unit 200 to be detachable/removable allows the control unit to be utilized with other neurostimulator configurations and also allows the brace 120 and the components remaining on the brace (e.g., the electrodes, etc.) to be replaced when worn out, expired, or otherwise due for replacement.

Advantageously, each stimulating electrode array 172 can be part of an assembly in which the stimulating electrodes 170 can be mounted on a substrate or housing 176 constructed, for example of plastic. This substrate/housing 176 can itself be secured to the brace 120 (e.g., via adhesives, stitching, or mechanical fastening) to thereby secure the stimulation electrode array 172 to be brace. Forming the stimulating electrode array 172 in this manner facilitates a precise arrangement and spacing of the stimulation electrodes 170 and makes it easy to secure them to the brace 120.

In a manner similar or identical to that of the example configuration of FIGS. 2A-D, the connector of each stimulating electrode array 172 can also be formed as a portion of the housing 176. The connector can be configured to protrude from a side of the housing 176 opposite the stimulation electrodes 170. The connector can, for example, extend through a hole in the brace 120 to position the connector on or extending from the outer surface 126. When the control unit 200 is connected to the connector, the brace 120 can be positioned between the control unit and the portion of the housing 176 supporting the stimulator electrode array 172.

Again, in a manner similar or identical to that of the example configuration of FIGS. 2A-D, the connector can support a plurality of terminals for electrically connecting the control unit 200 to the stimulation electrodes 170 and the recording electrodes 180. Certain terminals in the connector can be electrically connected to the stimulation electrodes 170 by wires or leads that are embedded within the plastic housing material (e.g., via insert molding). Embedding the leads in this manner helps maintain adequate spacing between the conductors, which avoids the potential for shorts in the circuitry.

Other terminals in the connector can be electrically connected to the recording electrodes 180 by wires or leads 184 that are partially embedded within the plastic housing material (e.g., via insert molding) and pass through the housing 176, extending to the recording electrode arrays 182. Through this configuration, all of the necessary electrical connections to the stimulation and recording electrodes 170, 180 are made when the control unit 200 is installed on the neurostimulator 110.

Referring to FIGS. 4C-D, the neurostimulator 110 is assembled by connecting the first and second portions 132, 134 of the upper portion 130 with the adjustment band 136. The upper and lower portions 130, 150 are interconnected by two adjustment bands 122 that interconnect their respective tab portions 144, 156. This completes the assembly of the neurostimulator 110, placing it in a condition to be worn by the subject in the manner illustrated in FIGS. 3A-B.

To use the neurostimulator 110, the brace 120 is simply unfolded and the control unit 200 is connected to the housing 176 via the connectors. The hook and loop fasteners 140, 142 and 152, 154 are disconnected, the brace 120 wrapped around the appropriate anatomy of the subject. In FIGS. 3A-B, the upper portion 130 is wrapped around the lower leg/ankle 112 of the subject, and the lower portion 150 is wrapped around the foot 114 of the subject. The hook and loop fasteners 140, 142 and 152, 154 are re-connected to attach neurostimulator 110 to the subject. Conveniently, where the neurostimulator 110 is configured for stimulating the tibial nerve in the position illustrated in FIGS. 3A-B, the upper portion 130 of the brace 120 can include visual alignment cues 210, such as holes in the brace, that become aligned with the medial malleolus of the ankle when the stimulating electrodes 170 are properly positioned.

Control Unit Configuration

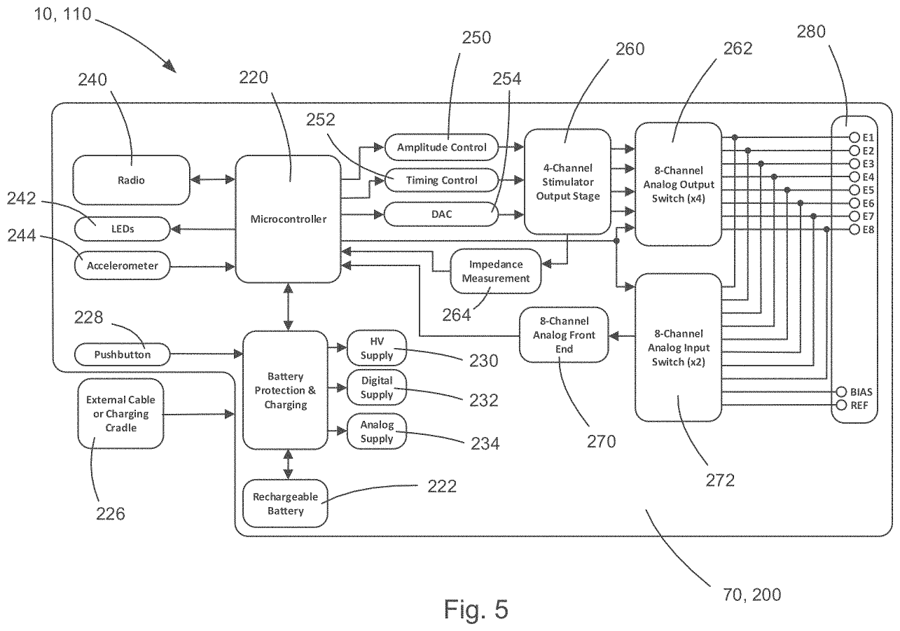

The control units 70, 200 of the example configurations of the neurostimulator 10, 110 of FIGS. 1A-4D can have a variety of configurations. An example configuration for the control units 70, 110 is shown in FIG. 5. Referring to FIG. 5, the control unit 70, 200 includes a microcontroller 220 powered by a primary or rechargeable battery 222 via a battery protection and charging circuit 224. The circuit 224 offers battery protection typical for a medical device, such as over-current and over-voltage protection, under-voltage protection, and a charging controller. An external cable or charging cradle 226 charges the battery 222 via the circuit 224. Alternatively, the battery 222 can be charged wirelessly, e.g., via a wireless charging cradle. A pushbutton 228 cycles on/off power to the control unit 70, 200.

The battery protection and charging circuit 224 also marshals power to a high voltage power supply circuit 230, a digital power supply circuit 232, and an analog power supply circuit 234. The high-voltage power supply circuit 230 is used to provide a stimulation compliance voltage to the output stage's current sources and sinks. Since this device is a transcutaneous stimulator, it can require a compliance voltage in the range of about 40-200 V or more in order to provide the necessary current to stimulate the tibial nerve. For this embodiment, a compliance voltage of 120 volts is used for the compliance voltage.

A radio controller 240, such as a Bluetooth® or Zigbee® radio controller, provides a communication input to the microcontroller 220 for functions such as programming the control unit 70, 200, uploading/downloading data, and monitoring/controlling the neurostimulator 10, 110 during use. The radio controller 240 could, for example, pair the microcontroller to an enabled device, such as a smartphone, tablet, or computer, executing software that enables the user to monitor or otherwise control the operation of the neurostimulator 10, 110. The microcontroller 220 controls the operation of indicators 242, such as LEDs, that indicate the state or condition of the control unit 70, 210. The microcontroller 220 can control an accelerometer 244, which can provide input to determine whether the neurostimulator 10, 110, and thus the subject, is moving or at rest.

The microcontroller 220 is responsible for controlling the stimulation output, measuring the electrode impedance, and processing the EMG response. The microcontroller 220 runs software for performing these functions, including decision-making algorithms to allow the device to provide the desired therapy. The microcontroller 220 controls the operation of an amplitude control circuit 250, a timing control circuit 252, and a digital-to-analog converter (DAC) 254. By “circuit,” it is meant that these functions can be implemented in any desired manner, e.g., through discrete components, integrated circuits, or a combination thereof. The amplitude control circuit 250, timing control circuit 252, and DAC 254 drive a stimulator output stage 260, which provides stimulator output signals (e.g., pulse-width-modulated “PWM” output signals) to one or more analog output switches 262. The output switch(es) 262 are operatively connected to a port 280 comprising a plurality of terminals (E1-E8 in FIG. 5) that facilitates connecting the control unit 70, 200 to the stimulator and recording electrodes, for example, via the leads 66, 184 (see, FIGS. 2A and 4B, respectively). Through this connection via the leads 66, 184, the stimulator output stage 260 can be operatively connected to the stimulator electrodes 50, 170.

The microcontroller 220 receives electrode impedance values via an impedance measurement circuit 264 that is operatively connected to the stimulator output stage 260. The microcontroller 220 also receives electrode feedback values (e.g., F-wave and M-wave values) via an analog front end 270 that is operatively connected to one or more analog input switches 272. The input switch(es) 272 are also operatively connected to the terminals/port 280 and can thereby receive feedback from the recording electrodes 60, 180 that facilitates connecting the control unit 70, 200 to the stimulator and recording electrodes, for example, via the leads 66 (see, FIG. 2A) or 184 (see, FIG. 4B).

The impedance measurement circuit 264 allows for measuring the impedance of the electrodes. It is important to measure the impedance often, in case one or more of the electrodes begins to lift from the skin. There are two potential hazards related to electrode lifting that should be mitigated. First, if an electrode is partially lifted from the skin, the surface area of the electrode that is in contact with the skin is reduced and the current density of the stimulation current is increased, which can be unsafe. Second, if an active electrode is completely lifted from the skin, a brief but large amount of energy can be delivered to the tissue when the electrode makes contact with the skin, which can result in pain.

Electrode impedances measured via the impedance measurement circuit 264 can also be used as an additional input for a closed-loop stimulation optimization algorithm.

The stimulator output stage 260 provides the current to the stimulating electrodes via the output switch 262. Each channel of the output stage includes a current source and current sink, which allows each channel to provide either a positive or negative current to the tissue through the corresponding stimulation electrode(s) 50, 170. In this configuration, each current source and sink can have independently programmable amplitude control 250 and timing control 252, which provides the capability to “steer” the current applied via the stimulation electrodes 50, 170, as described below. The programmable range can vary depending on the application, and is selected to be capable of achieving the desired nerve recruitment. In an example configuration, the current sources can have a programmable range from zero to +20 milliamperes (mA), and the current sinks can have a programmable range from zero to −20 mA.

As shown in FIG. 5, the analog output switches 262 and input switches 272 can both be operatively connected to each of the terminals E1-E8. Through operation of the switches 262, 272 as commanded by the microcontroller 220, the identity or role of the terminals, i.e., output terminal or input/feedback terminal, can be actively identified. This allows the microcontroller 220 to selectively identify, activate, and deactivate electrodes in a desired pattern, order, combination, etc., according to the particular therapy regimen being applied. This also allows the therapy to be tailored, for example, in response to signals received from the recording electrodes.

Control Overview

According to one example implementation, the neurostimulator 10, 100 described above can control the application of stimulation therapy according to two general phases: nerve localization and stimulation delivery. These two phases work synergistically to provide the functionality set forth in the following paragraphs.

During the nerve localization phase, the target peripheral nerve structure, e.g., the tibial nerve, is localized when the neurostimulator 10, 100 is donned and activated. In the nerve localization phase, the neurostimulator 10, 100 implements a process in which the following functions are performed:

Ramping up stimulation energy across various electrode patterns.

Monitoring EMG response after each stimulation pulse.

Determining the electrode pattern and stimulation parameters that optimally activate the target peripheral nerve.

During the stimulation delivery phase, electrical stimulation is delivered to the target peripheral nerve structure using the electrode pattern(s) and stimulation parameters determined during the nerve localization phase. In the stimulation delivery phase, the neurostimulator 10, 100 implements a process in which the following functions are performed:

Deliver stimulation pulses to the target peripheral nerve.

Continuously optimize the delivery of stimulation pulses, which includes:

Monitoring EMG response after each stimulation pulse.

Monitoring electrode impedance.

Adjusting either the electrode pattern (current-steering) or stimulation energy to optimize recruitment of the tibial nerve.

Automatically stopping stimulation at the end of the therapy session.

The nerve localization and stimulation delivery phases are described in more detail in the following sections.

Nerve Localization

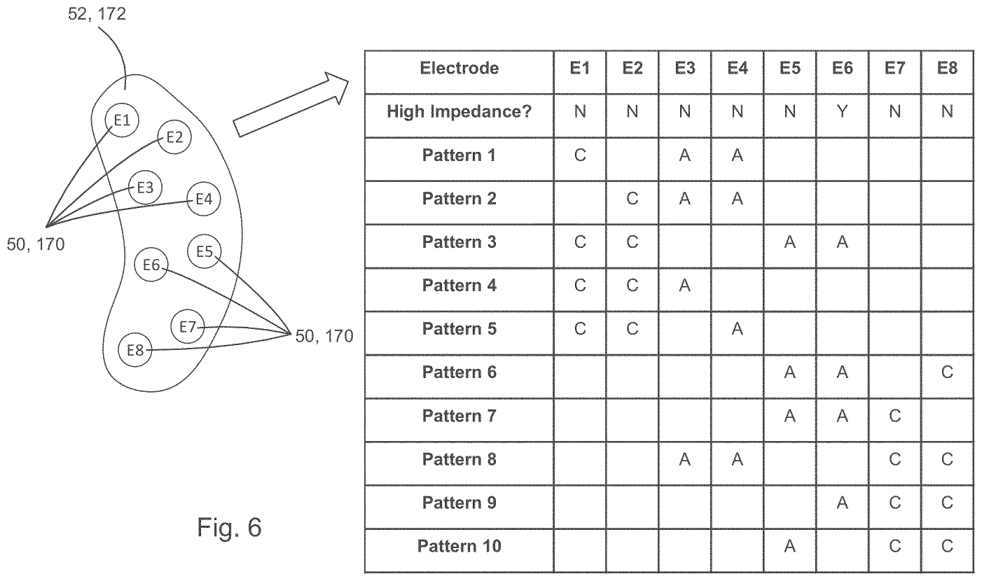

In practice, the control unit 110 can be programmed with a set of electrode patterns that identify which stimulation electrode 50, 170 in an electrode array 52, 172 are active, and also the polarity or type, i.e., anode (+) or cathode (−) assigned to the electrode. FIG. 6 illustrates an example configuration for an electrode array 52, 172 and a chart illustrating an example set of electrode patterns. In the example illustrated in FIG. 6, the electrode array 52, 172 has eight electrodes 50, 170, identified at E1-E8, and the chart identifies ten different electrode patterns (patterns 1-10) for the electrode array. For each electrode pattern, each electrode is identified as being a cathode (C), anode (A), or inactive (blank). Thus, for example, in pattern 3, electrodes E1 and E2 are cathodes, electrodes E5 and E6 are anodes, and electrodes E3, E4, E7, and E8 are inactive. While there are a large number of patterns that are possible with an eight-electrode array, the patterns can effectively be narrowed down to a shorter list, such as the illustrated 10 patterns or more, depending on the nerve under recruitment.

The neurostimulator 10, 110 can be configured to perform a nerve localization routine to determine which of the electrode patterns should be utilized on a subject. In the example configuration of FIG. 6, the electrode array 52, 172 can be specifically designed, i.e., shaped and electrodes positioned, to stimulate the tibial nerve in the region between the medial malleolus and the Achilles tendon. The electrode array 52, 172 can be configured to perform stimulation on this or other regions where peripheral nerve stimulation is desired.

In the example configuration of FIG. 6, the electrode array 52, 172 is curved to allow the medial malleolus to be used as a placement guide. Also, the array can be symmetrical so that it can be placed on either ankle. The electrode arrangement within the array must be configured to capture the tibial nerve, meaning that the nerve must pass below or between at least one pair of electrodes. If the tibial nerve passes outside the extents of the array, activation of the tibial nerve requires much higher stimulation energies, or it may not be possible to activate the tibial nerve at all.

The purpose of using an array for stimulation (as opposed to a single pair of electrodes) is to create an optimized stimulation field for recruiting the target (e.g., tibial) nerve. If the stimulation field is too small, the nerve will not be recruited and therapy will not be delivered. If the stimulation field is too large, too many motor neurons will be recruited resulting in undesired effects, such as pain, twitching, or muscle spasm. In order to optimize the stimulation field, the ability to steer current using multiple electrodes if preferred. For example, electrode pattern 8 assigns electrodes E3 and E4 as anodes and electrodes E7 and E8 as cathodes. Viewing the arrangement of these electrodes 50, 170 on the array 52, 172, it can be seen that the use of this electrode pattern could be effective on a nerve path that passes directly adjacent or between these electrode pairs.

By selecting the appropriate stimulation electrodes 50, 170 from the stimulation electrode arrays 52, 172, and varying the amplitude and polarity of the current applied via the selected electrodes, the electric field applied to the subject can be shaped so that the current is steered to the target nerves. By shaping the field, the neurostimulator 10, 100 can automatically adjust to day-to-day donning and placement variability for a given subject. Current steering also allows the neurostimulator 10, 100 to work across a subject population with wide anatomical variation, for example providing a shallow field for subjects with nerves that are superficial to the skin, or a penetrating field for subjects with nerves that are deep. In the illustrated example configurations, the stimulation electrode arrays 52, 152 include six electrodes. Any number of stimulation electrodes greater than one can be used. In general, the “field steering” capability of the neurostimulator 10, 100 increases with the number of stimulating electrodes 50, 170 that are included.

Because there will be session-to-session variability in the location of the stimulating electrode array 52, 172 due to the don/doff process, as well as variability in skin/tissue impedance, providing open-loop stimulation applying rigid pre-programmed stimulation parameters could be disadvantageous, often providing too little or too much stimulation energy to recruit the nerve. Advantageously, the nerve localization algorithm is executed at the beginning of each therapy session to determine which of the preprogrammed electrode patterns will be most effective.

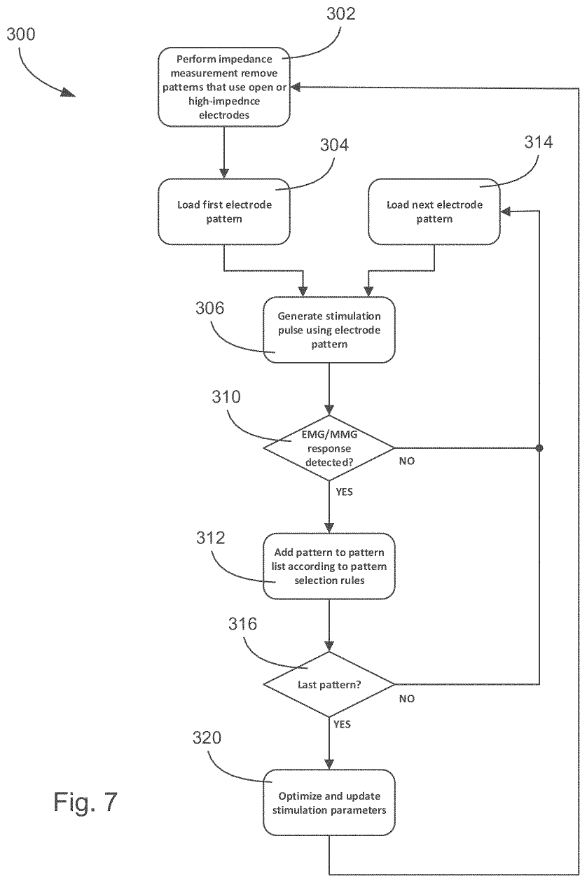

FIG. 7 illustrates a flowchart showing the method or process 300 implemented by the nerve localization algorithm. The steps in the process 300 are not meant to be exclusive, i.e., other steps can be included. Nor is the process 300 intended to be strictly followed in terms of the order shown in FIG. 7 or described herein. The process 300 illustrates steps, perhaps a minimum, necessary to localize the peripheral nerve that is to be stimulated.

It should be noted here that, the process 300 is a closed-loop algorithm that utilizes feedback recorded via the recording electrodes 60, 180 to make determinations and/or adjust settings. As such, the process 300 relies on utilization of the feedback to determine which of the electrode patterns effectively achieves nerve recruitment. Specifically, the process 300 relies on feedback from the recording electrodes 60, 180 to provide indication of EMG response feedback. Alternatively, the process 300 can rely on accelerometers to provide MMG response feedback.

Referring to FIG. 7, the process 300 begins at step 302, where an impedance measurement is performed in order to determine which, if any, of the electrodes E1-E8 have open or prohibitively high impedance. This step 302 can be considered an integrity check for the electrodes 50, 170 in the array 52, 172 to determine if any of the electrodes in the array are not sufficiently contacted with the skin. If any of the electrodes in the array are determined to be performing in a substandard manner, indicated by displaying an open (infinitely high) or sufficiently high impedance, those electrodes and the electrode patterns that utilize those electrodes can be eliminated from use.

For example, in the example of FIG. 6, it can be seen from row 2 that electrode E6 has high impedance. In this instance, electrode patterns 3, 6, 7, and 9 are eliminated form use in the current therapy session. Alternatively, the algorithm could instruct the control unit to provide some indication to the user, such as an alarm or display, to re-position or adjust the electrodes to see if contact can be improved.

To avoid interfering with stimulation and EMG measurement, the integrity check at step 302 can be completed in a short amount of time, such as 25 milliseconds or less. Also, the impedance measurement can be conducted so as to cause little or no sensation in the subject's skin. Therefore, the excitation current for performing the integrity check should be low-amplitude, such as 1 mA or less. For the integrity check 302, the impedance value at each electrode is not critical. Instead, determining whether the impedance is below a certain threshold is adequate.

Additionally, conditions other than high or low impedance can be determined in this integrity check. For example, indicators such as dry/wet contact checks, whole/brittle/fractured contact checks, contact surface area checks, and contact reflectance checks can be made during the connectivity evaluation. Sensors, such as don/doff, stretch, strain, bending or contact sensors (via electrical, optical or mechanical means) can also be used for conducting the connectivity evaluation. These sensors could also be incorporated into a buckle, clasp, snap, hook/eye or zipper feature.

Once the integrity check is performed, the process 300 proceeds to step 304 where the first electrode pattern (that hasn't been eliminated by the integrity check) is loaded. The process 300 then proceeds to step 306 where the neurostimulator 10, 110 generates stimulation pulse(s) using the electrode pattern loaded in step 304. The process 300 proceeds next to step 310, where a determination is made as to whether the stimulation pulses generated at step 306 elicited an EMG response, i.e., feedback measured via the recording electrodes. Step 310 can additionally or alternatively determine whether there is a MMG response where the feedback devices include accelerometer(s).

If, at step 310, EMG (or MMG) is not detected, the process 300 reverts to step 314, where a new electrode pattern is loaded. The process 300 then proceeds to step 306, as described above. If, at step 310, EMG (or MMG) is detected, the process 300 proceeds to step 312, where the electrode pattern is added according to pattern selection rules. The process 300 then proceeds to step 316, where a determination is made as to whether the current electrode pattern is the last electrode pattern in the list.

The pattern selection rules at step 312 for adding an electrode pattern can be defined to prioritize electrode patterns identified as being the best suited to recruit the target nerves. These pattern selection rules may be implemented as follows:

If one pattern is significantly better than the others (e.g., as determined from the EMG data, see below), that pattern should be used as the primary pattern moving forward.

If two or three patterns are roughly equivalent, any one of the patterns can be used as the primary pattern. Moving forward, this pattern can be switched to other ones if the nerve recruitment displayed by the current primary pattern begins to diminish.

If the nerve recruitment for a particular pattern begins to diminish and increasing the stimulation parameters does not fix the problem, similar patterns can be re-introduced to the algorithm.

If, at step 316, it is determined that the current electrode pattern is not the last pattern in the list, the process 300 reverts to step 314, where a new electrode pattern is loaded. The process 300 then proceeds to step 306, as described above. If, at step 316, it is determined that the current electrode pattern is the last pattern in the list, this indicates that the pattern list is complete. The process 300 proceeds to step 320 where the stimulation parameters for the electrode patterns in the pattern list are optimized. At step 320, the stimulation parameters (e.g., frequency, amplitude, pattern, duration, etc.) are updated to optimize the nerve recruitment for each pattern. The process 300 then reverts back to the initial step at 302 and proceeds as described above. If the recruitment for a given electrode pattern improves, the stimulation parameters are kept. If not, they revert back to previous values. This process repeats itself until the pattern list is filled with electrode patterns optimized for nerve recruitment.

From the above, it will be appreciated that the nerve localization process 300 determines which of the electrode patterns to utilize and which to discard for any given stimulation therapy session, and then optimizes the stimulation parameters for the utilized patterns. The execution of this process 300 is fast. During execution, the neurostimulator 10, 110 applies stimulation therapy pulses via the stimulating electrodes 50, 170 and monitors for EMG responses via the recording electrodes 60, 180 after each pulse.

The analog front end circuit 270 can replace traditional EMG measurement circuitry such as a filter, amplifier, rectifier, and/or integrator. The control unit 110 utilizes the analog front-end circuit 270 to sample the recording electrodes at a predetermined sample rate, such as 1,000-8,000 samples per second. The EMG sampling window will begin after the stimulation pulse is finished, and the window will last for a predetermined brief period, such as 8-90 milliseconds. The resulting EMG data, comprised of M-wave or F-wave or both, will be analyzed using a Fast Fourier Transform (FFT) technique that clearly shows if EMG is present.

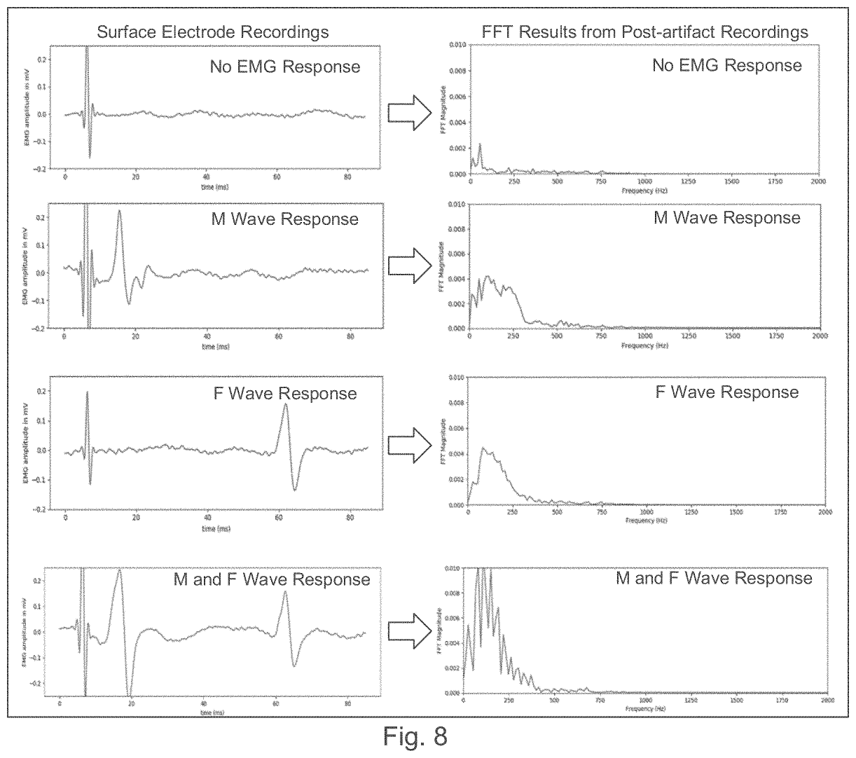

To execute the process 300 of FIG. 7, the neurostimulator 10, 110 monitors for electromyogram (EMG) signals via the recording electrodes 60, 180 in response to stimulation applied via the stimulation electrodes 50, 170. FIG. 8 illustrates examples of the EMG responses that can be recorded, which include: No EMG Response, F-wave Response, M-wave Response, and M and F-wave Response. In the example where no EMG response is recorded, the stimulation pulse artifact can be seen on the left, with no response following. In the example where an M-wave response is recorded, the stimulation pulse artifact can be seen on the left, followed by the M-wave at about 6 to 10 ms post-stimulation. In the example where an F-wave response is recorded, the stimulation pulse artifact can be seen on the left, followed by the F-wave responses at about 50 to 55 ms post-stimulation. In the example where both an M-wave and F-wave responses are recorded, the stimulation pulse artifact can be seen on the left, followed by the M-wave and F-wave at 6 to 10 ms and about 50 to 55 ms post-stimulation, respectively. These response times could change slightly, depending on a variety of factors, such as the hydration and/or salinity of the subject tissue, the arrangement and spacing of the electrodes, and the characteristics of the stimulation signals.

For each of the four recorded response scenarios, FIG. 8 also illustrates a corresponding Fast Fourier Transform (FFT) results for the raw post-artifact signal. The FFT results are calculated by the microcontroller 220 and are used in the process 300 to determine whether an EMG response is present (see, step 310 in FIG. 7).

### Stimulation Delivery

The neurostimulator 10, 110 can apply stimulation therapy using an open-loop control scheme, a closed-loop control scheme, or a combination of open-loop and closed-loop control schemes, depending on the control algorithm programmed into the microcontroller 220. For open-loop control, the control units 70, 200 can apply electrical stimulation via the stimulation electrodes 50, 170 according to settings (frequency, amplitude, pattern, duration, etc.) without regard to any feedback measured via the recording electrodes 60, 180. This is not to say that feedback is not measured, just that, in an open-loop control scheme, the feedback is not used to inform or control the algorithm executed by the microcontroller 220 to control the application of stimulation therapy. In a closed-loop control scheme, the neurostimulator 10, 110 implements a control algorithm in which feedback from the recording electrodes 60, 180 informs and helps control the application of stimulation therapy.

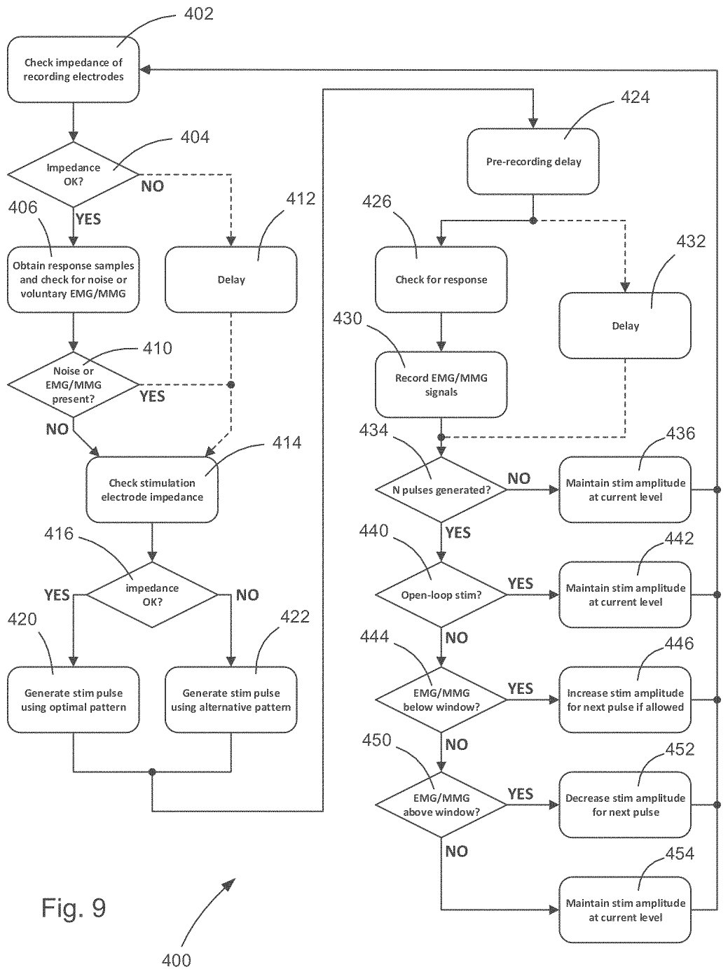

FIG. 9 illustrates by way of example a process 400 by which the neurostimulator 10, 110 controls the application of electrical nerve stimulation using the electrode pattern(s) identified by the nerve localization process 300 of FIG. 7. The stimulation control process 400 can employ both open-loop and closed-loop control, with closed-loop steps or portions of the process being illustrated in solid lines and open-loop steps or portions being illustrated in dashed lines. Ideally, the process 400 will proceed with closed-loop control, as it is able to utilize feedback to optimize the application of stimulation therapy.

The process 400 begins at step 402, where the impedances of the recording electrodes 60, 180 are checked. If, at step 404, it is determined that the recording electrode impedances are too high (e.g., resulting in unavailable or unreliable feedback), the process 400 then shifts to open-loop mode (see dashed lines) and proceeds to step 412, where a delay is implemented. The purpose of delay 412 is to assist in maintaining a constant stimulation period, meaning that the duration of delay 412 should be equal to the duration of closed-loop step 406. After completing delay 412, the process 400 proceeds to step 414, where the stimulation electrode impedances are checked.

At step 404, if the impedances of the recording electrodes are acceptable, the process 400 remains in closed-loop mode and proceeds to step 406, where samples are obtained via the recording electrodes to check for significant noise or voluntary EMG responses. At step 410, if noise or EMG are present, the feedback is considered unreliable and the process 400 shifts to open-loop mode and proceeds to step 414. At step 410, if significant noise or voluntary EMG is not present, the feedback is considered reliable and the process 400 remains in closed-loop mode and proceeds to step 414.

At step 414, regardless of whether the process is in open-loop mode or closed-loop mode, the impedances of the stimulation electrodes 50, 170 are checked. At step 416, if the stimulation electrode impedances are acceptable, the process 400 proceeds to step 420 and the neurostimulator 10, 110 generates stimulation pulses, which are applied via the stimulation electrodes using the optimal electrode pattern, as determined by the nerve localization process 300 (see FIG. 7). If, at step 416, the stimulation electrode impedances are too high, the process 400 proceeds to step 420 and the neurostimulator 10, 110 generates stimulation pulses that are applied via the stimulation electrodes using an alternative electrode pattern selected from the pattern list determined by the nerve localization process 300. In either case, after generating the stimulation pulse using the optimal pattern (step 420) or the alternative pattern (step 422), the process 400 proceeds to step 424.

At step 424, the process 400 implements a pre-recording delay to allow time for the electrical stimulation applied at step 420 or 422 to elicit an EMG response. As discussed above, these delays can be relatively short, so the delay at step 424 can, likewise, be short, e.g., 5 ms or less. If the process 400 is in open loop mode, it proceeds to step 432, where a further delay is implemented. This delay 432 should match the duration of closed-loop steps 426 and 430 so that a constant stimulation period is maintained. If the process 400 is in closed-loop mode, it proceeds to step 426 and checks for feedback via the recording electrodes 60, 180. The process 400 then proceeds to step 430, where any detected EMG feedback signals are recorded and analyzed.

At this point, regardless of whether the process 400 is in open-loop mode (step 432) or closed-loop mode (step 430), the process proceeds to step 434, where a determination of whether the number of stimulation pulses applied in the current therapy session has reached a predetermined number (N). If the predetermined number (N) of pulses have not yet been applied, the process proceeds to step 436, the stimulation amplitude is maintained at the current level, and the process 400 reverts back to step 402, where the impedance of the recording electrodes is checked and the process 400 repeats. If, at step 434, the predetermined number (N) of pulses has been reached, the process 400 proceeds to step 440.

At step 440, if the process 400 in open-loop mode, the process proceeds to step 442, the stimulation amplitude is maintained at the current level, and the process 400 reverts back to step 402, where the impedance of the recording electrodes is checked and the process 400 repeats. At step 440, if the process 400 is not in open-loop mode (i.e., is in closed-loop mode), the process proceeds to step 444, where a determination is made as to whether the EMG recorded at step 430 is below a predetermined window, i.e., below a predetermined range of acceptable EMG values. If the EMG is below the predetermined window, the process 400 proceeds to step 446, where the stimulation amplitude is increased for the next pulse, if permitted. The process 400 then reverts back to step 402, where the impedance of the recording electrodes is checked and the process 400 repeats with the increased stimulation amplitude.

If, at step 444, the EMG is not below the window, the process 400 proceeds to step 450 where a determination is made as to whether the EMG is above the predetermined window. If the EMG is above the predetermined window, the process 400 proceeds to step 452, where the stimulation amplitude is decreased for the next pulse. The process 400 then reverts back to step 402, where the impedance of the recording electrodes is checked and the process 400 repeats with the decreased stimulation amplitude. If, at step 450, the EMG is not above the predetermined window, the EMG is determined to be within the predetermined window and the process 400 proceeds to step 454, where the stimulation amplitude is maintained at the current level for the next pulse. The process 400 then reverts back to step 402, where the impedance of the recording electrodes is checked and the process 400 repeats.

### Elongated Electrodes for Monitoring of EMG by Simultaneous Recruitment of Multiple Muscles

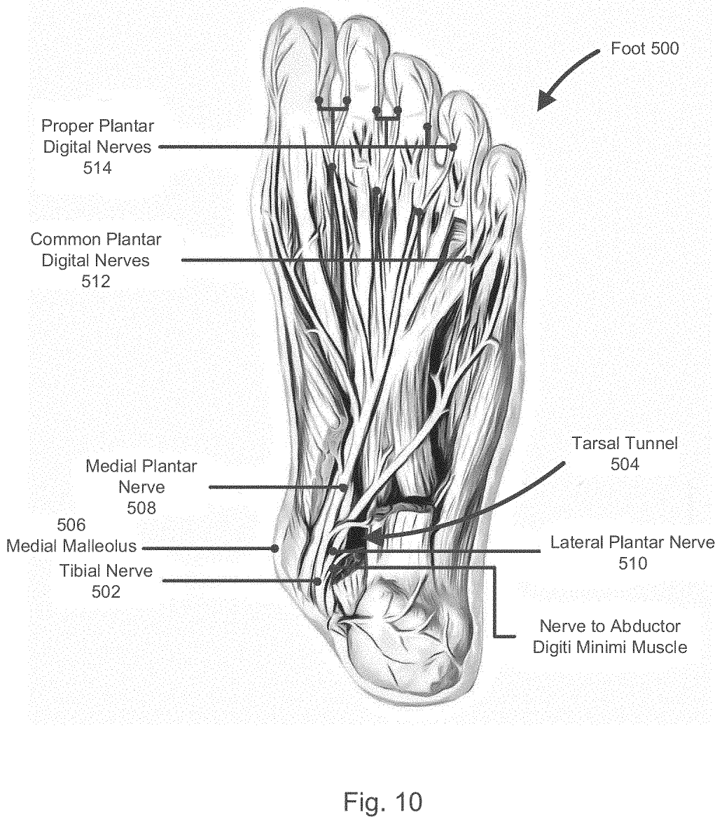

FIG. 10 illustrates the primary innervation of the human foot 500. The tibial nerve 502 travels inside the foot 500 via the tarsal tunnel 504, posterior towards the medial malleolus 506. The tibial nerve 502 lies lateral towards the posterior tibial artery inside the tarsal tunnel 504 and also produces medial calcaneal branches, in order to innervate the heel while penetrating the flexor retinaculum. The tibial nerve 502 bifurcates with the posterior tibial artery, in the middle of the medial malleolus and the heel, into a large medial plantar nerve 508 and a smaller lateral plantar nerve 510. The plantar nerves 508, 510 branch into the common plantar digital nerves 512 and the proper plantar digital nerves 514.

As discussed previously, stimulation of nerves, such as the tibial nerve 502, can provide therapeutic benefits to multiple conditions, with one example being overactive bladder (OAB). For consistent therapy, monitoring muscle activity induced by the activation of neuromuscular junction is important. For example, the neurostimulator 10, 110 described above with reference to FIGS. 1-9 includes electrodes 60, 180 for monitoring Electromyography (EMG) from the post-synaptic muscle, which allows to confirm pre-synaptic nerve recruitment, as well as adjust stimulation parameters to provide optimized therapy levels. These electrodes can be built into a wearable garment, thus precluding the need for manually placement of the electrodes as a separate part of the system for each therapy session.

FIGS. 11 and 12 illustrate example configurations of neurostimulator designs that can implement recording electrodes that provide a robust and reliable signal under a broad variety of conditions, such as anatomical differences between the subject wearing the device, differences in placement of the electrodes on the subject, variability in the position of the stimulator garment on the subject, relative movement or shifting of the recording electrodes relative to the target muscle groups, physical bodily movement during use, and undulations in the foot profile. The improved recording electrodes can facilitate therapy that is uninterrupted during normal daily activities, which can significantly improve the usability and compliance of the system.

FIG. 11 illustrates a neurostimulator 520 that is generally similar in design and operation to the neurostimulator 110 of FIGS. 3A-4C, with the exceptions described below. The neurostimulator 520 has a brace configuration including a brace 522 upon which the neurostimulator components are supported. The configuration of the neurostimulator 520 is similar in some respects, and identical in others, to the braced configuration shown in FIGS. 3A-4C. The neurostimulator 520 can thus be worn as a garment in the manner shown in FIGS. 3A-B.

FIG. 12 illustrates a neurostimulator 550 that is generally similar in design and operation to the neurostimulator 10 of FIGS. 1A-2E, again with the exceptions described below. The neurostimulator 550 has a strap configuration including a strap 552 upon which the neurostimulator components are supported. The configuration of the neurostimulator 550 is similar in some respects, and identical in others, to the strap configuration shown in FIGS. 3A-4C. The neurostimulator 550 can thus be worn in the manner shown in FIGS. 1A-B.

The manner in which the neurostimulators 520, 550 are supported on the subject, i.e., worn, can vary. For example, the neurostimulators could be configured in the form of a sock that fits over the subject's foot and ankle, or in the form of a sleeve that slides over the foot/ankle, leaving the toes exposed. The support structure for positioning the neurostimulator components on the subject can have any configuration suited to place the components at the desired location on the subject.

The neurostimulators 520, 550 include recording electrodes 524, 554, respectively, that have an elongated profile configured to extend laterally across the longitudinal muscle groups of the foot (see, FIG. 10). This relieves the need to focus the monitoring of EMG feedback on a specific post-synaptic muscle being activated. The recording electrodes 524, 554 cover a large anatomical area of the foot so as to record activation of muscle tissue that is located adjacent or near the electrodes. This helps minimize the likelihood of a total loss of electrical evoked muscle signals, compared to recording electrodes that rely on a more precise placement.

The neurostimulators 520, 550 can be configured so that the elongated recording electrodes 524, 554 span over the whole width of the bottom of the foot 500. This is shown in FIG. 13. The recording electrodes 524, 554 can alternatively be configured to have lengths to provide different coverage of the foot 500, and can also be configured to be positioned in alignment with each other, or staggered relative to each other, so that at least one of the electrodes covers the entirety of the target muscle bundles. The spacing between the recording electrodes 524, 554 can, for example, be between 6 cm and 12 cm, measured from the longitudinal centerlines of the electrodes, as indicated generally at dimension X in FIG. 13.

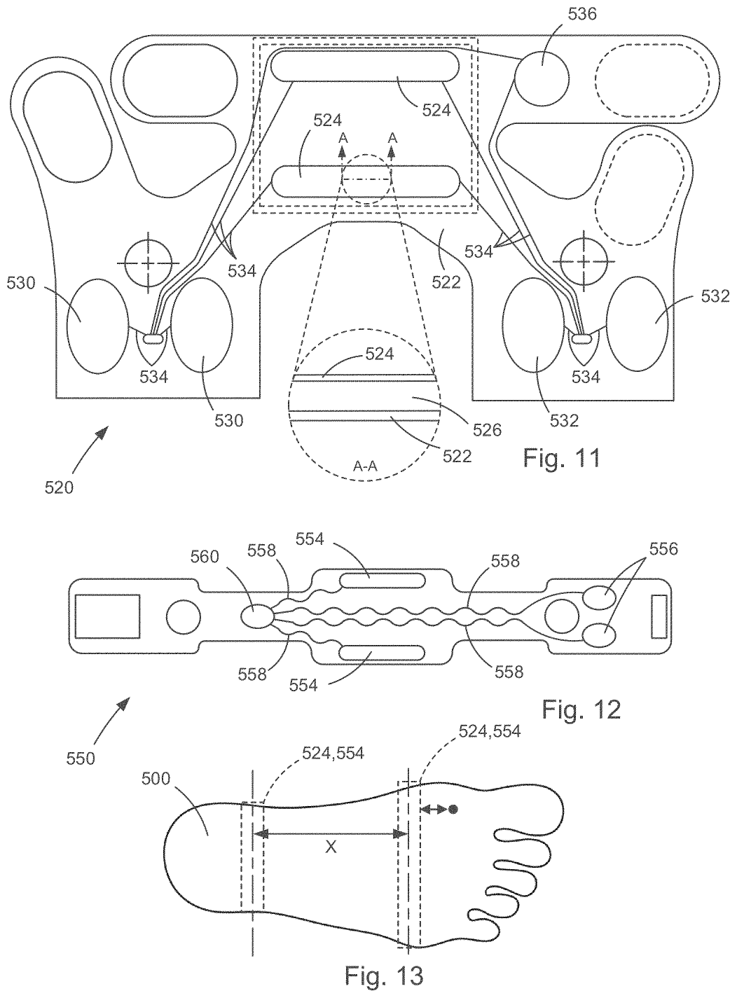

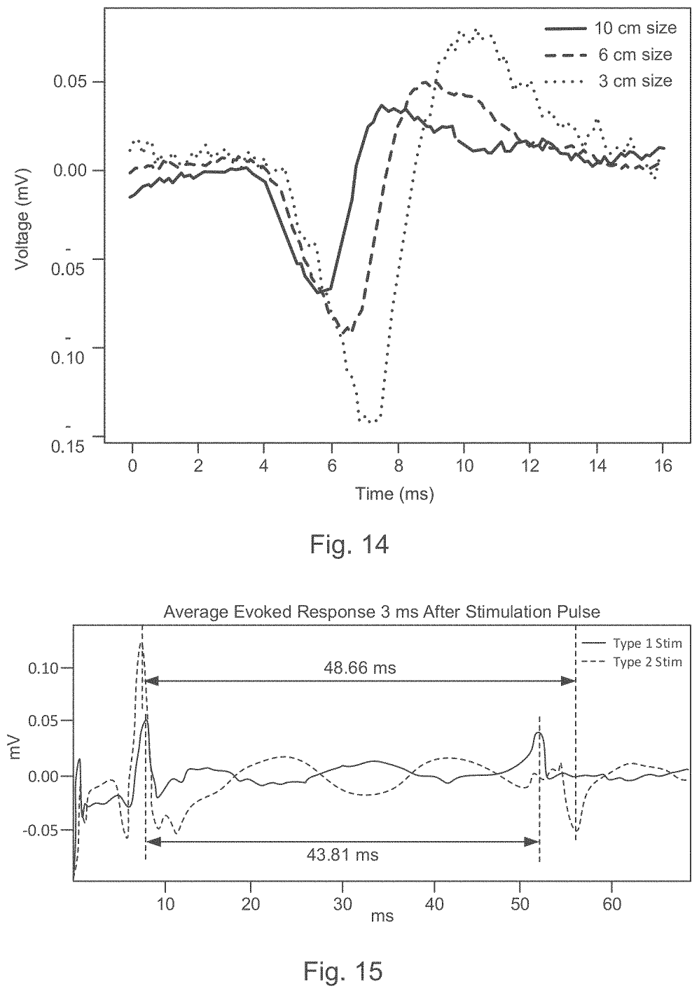

FIG. 14 illustrates the effect that the size, i.e., width of the recording electrodes 524, 554 has on the voltage recorded in response to stimulation applied in an identical manner. As shown in FIG. 14, all three size—3 cm, 6 cm, and 10 cm, recorded a response, and the response had a similar waveform. The amplitudes of the recorded responses varied inversely with the size of the recording electrodes. This shows that the large electrodes 524, 554 are capable of recording EMG responses to tibial nerve stimulation.

When more than one muscle is recruited, it has been confirmed that there is no adverse impact on the integrity of the combined feedback signal received by the elongated electrodes 524, 554 due to their simultaneous recruitment. This feedback signal is further analyzed using particular signal processing and noise reduction techniques. The elongated electrodes 524, 554 can therefore advantageously improve the recording function of the neurostimulators 520, 550.

To promote good, reliable contact between the electrodes and the subject's foot, the neurostimulators can include a compliant member that facilitates forming the electrodes to the contour of the foot. This is shown by way of example in the magnified section view detailed in FIG. 11. In one embodiment of the system, a compliant member is added underneath the recording electrodes, to accommodate different foot profiles and potential undulations. Such a compliant member could be a part of the garment, such as a sheath of foam or silicone embedded in the fabric, or it could be an external wearable system, such as a band, that essentially provide a similar and uniform pressure on the recording electrodes. The form and stiffness of this compliant member may be customized based on individual size or the arch of the foot.

Integrated Wearable Device with Built-In Stimulating and Recording

The neurostimulators described herein, including the neurostimulators 520, 550 of FIGS. 11 and 12, can have an integrated construction in which the stimulating and recording elements, e.g., electrodes, traces, etc., are integrated into a single wearable garment. This construction ensures the positioning of the elements on the garment which, in turn, ensures the automatic placement of all the electrodes when the garment is worn by the subject.

FIGS. 11 and 12 illustrate examples of components that can be integrated with the neurostimulator garments. Referring to FIG. 11, the stimulating electrodes 530, 532 and recording electrodes 524 electrically connected to conductive traces 534, which provide the electrical connectivity to the controller (not shown) via connector 536 (shown schematically). Similarly, referring to FIG. 12, the stimulating electrodes 556 and recording electrodes 554 electrically connected to conductive traces 556, which provide the electrical connectivity to the controller (not shown) via connector 560 (shown schematically). FIGS. 11 and 12 are, of course, examples of the types of neurostimulators into which this integrated construction can be implemented. It will be appreciated that the integrated construction can be implemented in various alternative neurostimulator configurations, including any of the configurations disclosed herein.

The neurostimulators 520, 550 have integrated constructions in which the electrodes and traces are embedded into their respective garments 522, 552, thus eliminating a need for external wiring, adhesive or other such mechanisms that can limit the usability or reliability of the garment. According to one implementation, the stimulation electrodes, recording electrodes and traces are all fabricated as a single part in which the electrically conductive and insulating components are formed as one or more layers of electrically conductive materials, such as a flexible printed circuit, that is supported on a flexible substrate.

This prefabricated part may than be attached to the garment using a multiplicity of processes, one such example being thermal pressing. In this construction, the substrate supporting the electrical components can comprise a thermal adhesive that facilitates the thermally pressed attachment. Alternatively, the conductive and insulative layers can be directly imparted on the garment using processes such as spraying or deposition.

The electrodes have conductive material exposed to ensure good contact with patient body. The traces may be made from a conductive material printed on a non-conductive sheet and then adhered to the garment. However, an electrical contact between the traces and human body is undesirable, and prevented by means of insulation, which could be the non-conductive sheet, or may include an additional layer of insulation material. The garment may be made of a material that provides sufficient flexibility, is compatible with human body and allows for electrode printing. An example of such garment material may be neoprene. Thus, a system having all electrodes and traces within a single component minimizes any connectivity losses, compatibility or dimensional tolerancing challenges.

Advantageously, these constructions have the ability to flex during normal use of the garment when the fabric is stretched. To facilitate stretching, the traces can be configured to have a curved/bent/waved appearance, as shown with the traces 558 in the example configuration of the neurostimulator 550 of FIG. 12. When the garment 552 is stretched, the curved traces 558 can un-curve/un-bend so that the electrical continuity of the traces is maintained. This curved/bent/waved configuration of the electrical traces can be implemented in any of the neurostimulators disclosed herein.

### Method of Automatic Detection of Sidedness of Garment on a Human Subject

According to another aspect of the invention, the neurostimulators described herein can be configured to automatically detect the foot, i.e., right or left, upon which the neurostimulator is worn. The neurostimulator is configured to be worn on either foot. Regardless of the foot upon which the neurostimulator is worn, the recording electrodes are positioned across the foot in the manner shown in FIG. 13. The stimulating electrodes, however, positioned on the ankle at the tibial nerve near the medial malleolus, are positioned differently depending upon which foot, right or left, the neurostimulator is worn.

Advantageously, since the recording electrodes 524, 554 extend across the foot (see FIG. 13), there is no need to have recording electrodes 520, 550 that are specific to a left or right foot implementation. For the two primary garment types disclosed herein (H-brace 520—FIG. 11 and strap 550—FIG. 12), the stimulation electrode arrangements are mirror imaged so that the neurostimulators can be worn on either foot. Specifically, the H-brace neurostimulator 520 (FIG. 11) includes left stimulating electrodes 530 and right stimulating electrodes 532. When worn on the left foot, the left stimulating electrodes 530 are positioned on the left ankle at the tibial nerve near the medial malleolus. When worn on the right foot, the right stimulating electrodes 532 are positioned on the right ankle at the tibial nerve near the medial malleolus.

The strap neurostimulator 550 (FIG. 12) can include a singular set of stimulating electrodes 556. This is because the strap 552 is symmetrical and can be flipped too position the stimulating electrodes 556 on the ankle at the tibial nerve near the medial malleolus for the left or right foot. In this scenario, however, since the neurostimulator 550 is flipped, both the recording electrodes 554 and the stimulation electrodes 556 are also flipped from front to back and vice versa. Because of this, depending on the foot upon which the foot is worn, the electrodes 554, 556 will be located on the front on one foot, and on the rear on the other foot. Similarly the stimulation electrodes 556 reverse polarity, such that the electrode that was cathode on one foot becomes the anode on the other foot.

The neurostimulators 520, 550 are configured to record the evoked muscle response to the activation of tibial nerve as a phase relationship (or time delay) between the stimulation signal and the EMG response. When the garment is moved from one foot to the other, this phase relationship is altered, thus providing a unique differentiator between the two feet. The phase relationship is shown in FIG. 15. In FIG. 15, the average evoked response 3 ms after a stimulation pulse is shown for two types of stimulation identified as Type 1 and Type 2. Types 1 and 2 are simply the same stimulation pulse applied on a different foot of the same subject. As shown in FIG. 15, the evoked response from the stimulation pulse differs depending on the foot upon which it is applied. Through clinical calibration, this phase relationship can be correlated with each foot, thus providing a unique identification of which foot the garment is worn on. By programming the controller of the neurostimulators 520, 550 with these unique identifications, the foot onto which the neurostimulator is fitted can be determined automatically without input from the user. This determination can be used to select the polarity of the stimulating electrodes in the strap configuration of the neurostimulator 550, or can be used to select which set of stimulating electrodes—left 530 or right 532—to use.

In another configuration of the neurostimulator 520, 550, the need to switch electrode polarity in response to the foot onto which the device is fitted can be avoided. In this configuration, the neurostimulator 520, 550 can be configured to include redundancy in stimulation electrodes. For the H-brace neurostimulator 520, the redundancy is shown in the left/right electrodes 530, 532. For the strap neurostimulator 550, the redundancy can be implemented by altering the pin configuration to selectively chose a pair (or group) of electrodes. To make this determination, the controller is configured to alter the pin configuration of the neurostimulator to alter the measured impedance between the stimulation electrodes. The left/right foot determination is made by finding the impedance between the electrodes that is indicative of the foot location. In one implementation, the expected impedance can be about 5k-ohm.

In a further configuration, the spacing between the cathode and anode may be deliberately made unequal between Left and Right side of the garment. This will result in two differences. First, the overall feedback signal, including phase and amplitude, will be different because the response is dependent on stimulation electrode configuration and spacing. Second, this will cause the impedance between the two electrodes to be different. Either of these values can be measured during the therapy session, and thus can then be used to determine which foot of the subject.

### A System of Providing Optimal Charge for Neurostimulation

As discussed previously, the neurostimulators 520, 550 have wide therapeutic applications, such as pain management and bladder control. According to these treatment methods, a known amount of charge is applied through either a pair or multiplicity of electrodes attached to the subject's body. Most systems determine the amount of charge using the amplitude of the voltage or current applied, or through the duration of the pulse, or pulse width, of the voltage of current applied. All these methods have limitations in terms of therapy range, energy usage and in accounting for different patient sensation or anatomical response.

According to another feature, the neurostimulators 520, 550 can be configured to control the application of stimulation therapy in a manner that compare the amplitude of the stimulation signal to the pulse width, to provide a optimal combination of therapy, energy use, patient sensation and ease of use. This can be implemented in both closed-loop, with where stimulation is modulated based on an evoked electrical response, or in open-loop where no response is recorded. Also, the neurostimulators 520, 550 can be configured for current-control or voltage-control. Because of this, it should be understood that, when the term ‘stimulation signal’ is used herein, it can be associated with electric current or voltage.

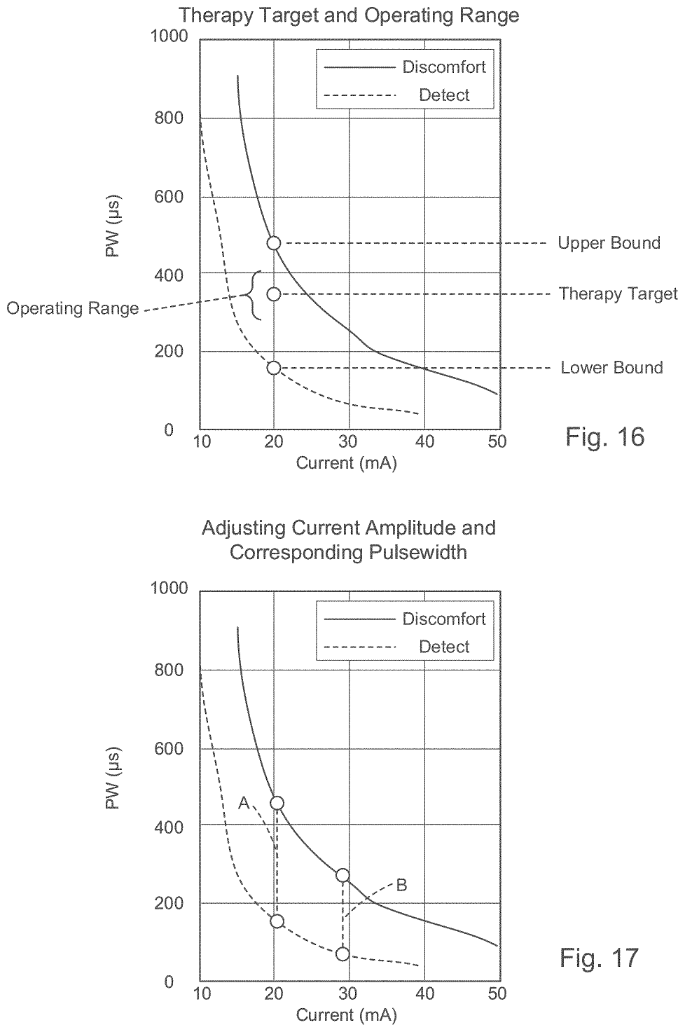

In one example configuration, a method for determining optimal charge for neurostimulation involves applying stimulation within a range of pulse widths that are defined by both the subject's tolerance as well as the threshold for evoking a response. This is shown in FIG. 16. In this example, a closed-loop current-controlled system adjusts the pulse width up or down based on the EMG response feedback signal measured via the recording electrodes. The upper bound of the pulse width can be defined at or near the patient's tolerance limit, as shown by the solid dicsomfort line shown in FIG. 16. The lower bound of the pulse width can be defined at or near the threshold for evoked response. In the example of FIG. 16, the target therapy is determined at a certain point, between these two parameters, such as the midpoint, and the therapeutic range is determined to be a fraction of the target therapy level, as indicated generally by the bracket in FIG. 16.

After the initiation of therapy and over the course of time, a need to change the therapeutic regime can arise. this can result, for example, from a patient's tolerance changing over time, device characteristics changing over time, or the body's response changing as a result of therapy. Accordingly, the applied current amplitude can be adjusted and a new corresponding range of pulse width defined. This is shown in the example of FIG. 17. As shown in FIG. 17, as an example, patient discomfort and detection thresholds may define an initial current of 20 mA (shown at A) with a corresponding range of pulse widths. Over time, however, for one or more of the reasons set forth above, a higher stimulation charge may be desired. Accordingly, for example, the current amplitude can be manually increased to 30 mA (shown at B), defining a correspondingly new operating range for the pulse width. The difference between the curves in FIG. 17 define between them a region that defines a range of stimulation strength-duration curve for a sample subject.

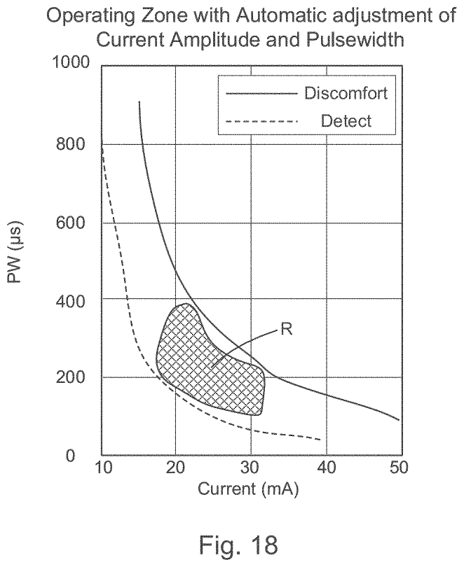

As another example configuration, stimulation can be executed within an operating zone defined by a range of pulse widths and range of current amplitudes. This is shown in FIG. 18. As shown in FIG. 18, these ranges are illustrated by the shaded region R, which defines the operating parameters, pulse width and current amplitude, according to which stimulation therapy is executed. Operating within the defined range allows the controller to adjust both the current amplitude and pulse width individually or simultaneously. The controller can operate in closed-loop mode using EMG feedback to modulate the current and pulse width, as described previously. Alternatively, the controller can operate in closed-loop mode using stimulation energy as the feedback, with the tolerance limits of the subject being used to help determine setpoints for the energy, and the stimulation output is modulated to maintain that energy level setpoint. These parameters, i.e., tolerance limits and corresponding energy setpoints, can be defined during the initial calibration, and they system makes the decisions on the current amplitude and pulse width based on this calibration, while delivering the desired stimulation charge.

### Providing Optimal Therapeutic Control Parameters for Neurostimulation, Based on Patient's Motor and Neural Response.

Stimulation of nerves has wide therapeutic applications, such as pain management or bladder control. For best possible patient outcomes, it is important to determine the optimal stimulation parameters that provide therapeutic benefits, while ensuring no patient discomfort that could lead to non-compliance. Accordingly, a method for determining these optimal stimulation parameters utilizes multiple factors, including patients' muscle and sensory responses. According to the method, the therapy target is based on the individual patient's response induced by the stimulation, therapeutic needs and tolerance threshold, while at the same time ensuring the therapeutic window never extends beyond any of these limits.

According to this method, the closed-loop system is employed that detects and quantifies the stimulation evoked response, such as EMG or nerve response, when a stimulation is applied. The lower threshold of therapeutic window is defined at the level at which the evoked response is detected. This is based on two factors, one being a physical confirmation of recruitment of the corresponding nerve to ensure system operates as intended, and second being the ability to continuously adjust the stim based on the evoked response. The upper threshold is defined by the sensory feedback, or at a level that a patient can comfortably tolerate for a duration of a typical therapy session.

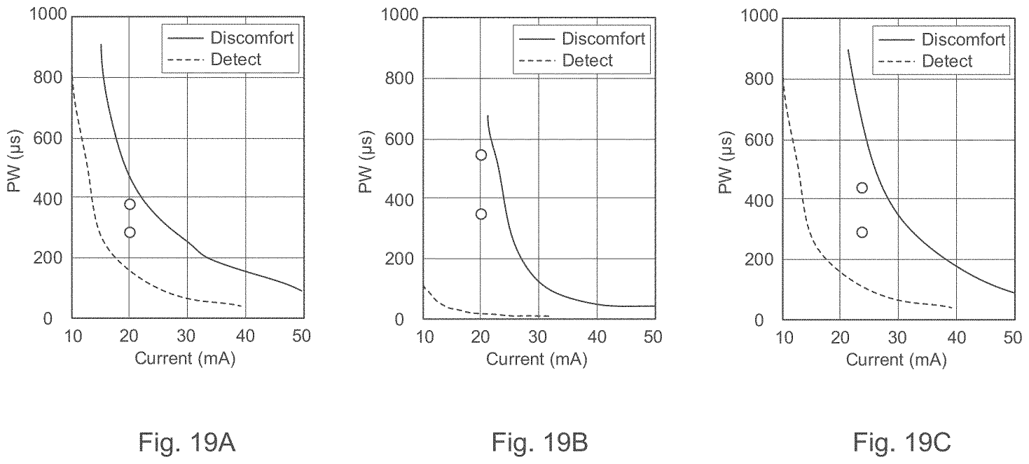

The upper (discomfort) and lower (detection) thresholds define the operating range and also define the optimal stimulation therapy that is targeted for a specific patient. This patient-specific target therapy is linearly interpolated between the upper and lower thresholds in a manner that is determined by the clinical need for a certain indication. Examples of these interpolated target therapy ranges are illustrated in FIGS. 19A-19C. Referring to FIGS. 19A and 19B, stimulation current is constant at 20 mA with the pulse width being modulated to apply therapy between the discomfort and detection thresholds. In this example, the linear interpolation can be at the midpoint, such that low end of the stimulation pulse width range is at 50% of the range and the upper end is at 75% of the range. Comparing FIGS. 19A and 19B, it can be seen that the detection and discomfort thresholds, which are patient-specific, determine the upper and lower limits of the 50-75% pulse width range. While this example illustrates a 25% range, alternative ranges, higher or lower, can be implemented.

Alternative ranges can be selected, for example, to increase the system output. To achieve this, the lower limit can, be defined at a higher percentage of the range, such as 75% of the range. In this example, the upper range can be set accordingly, such as at 85-90%. As shown in FIG. 19C, it can be seen that the stimulation current also can affect the upper and lower limits of the 50-75% pulse width range. Increasing the current moves the range to the right, as shown in FIG. 19C, where the threshold curves have reduced pulse widths. The pulse width range is therefore reduced accordingly at this higher stimulation current.

The examples of FIGS. 19A-19C utilize variable pulse width at a fixed current amplitude. Alternatively, a range determination may be made for systems that use a fixed pulse width and variable current amplitude. Furthermore, a system can comprise of a combination of variable current and pulse width, for example to optimize power consumption, and a target may similarly be obtained based on the amount of charge applied through stimulation.

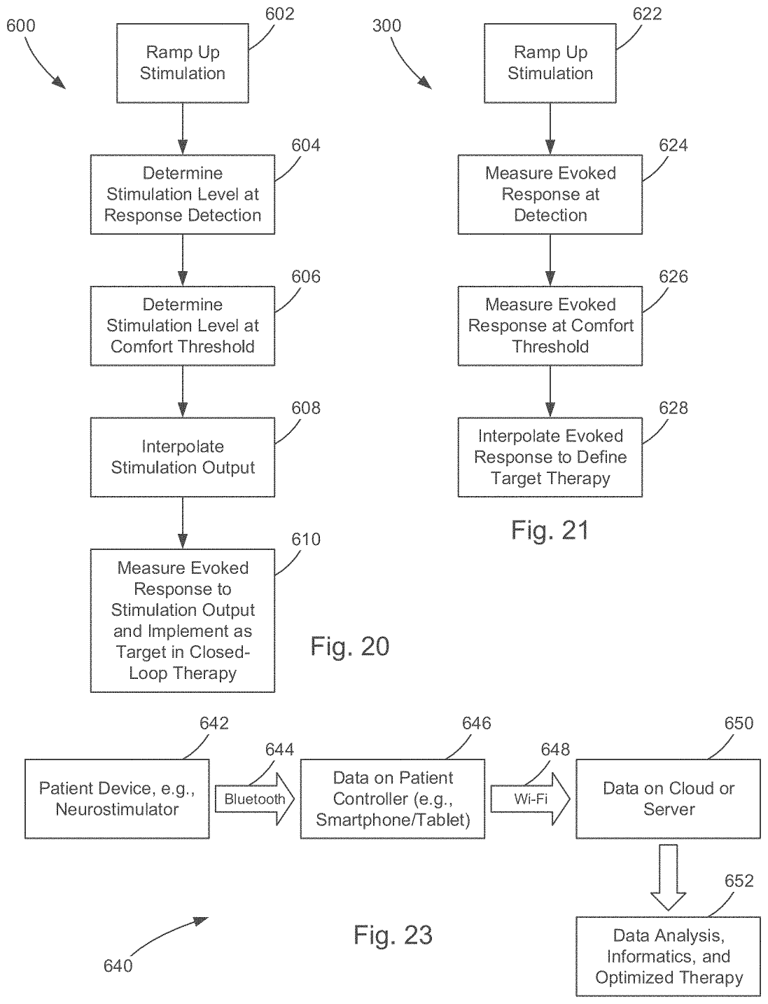

FIGS. 20 and 21 illustrate two different methods by which the target stimulation is determined. According to the method 600 of FIG. 20, at step 602, stimulation is ramped up, i.e., the pulse width is increased at a constant current amplitude. At step 604, the detection stimulation level (i.e., where a response, such as EMG, is detected) is determined. At step 606, the discomfort stimulation level (i.e., where the subject experiences discomfort) is determined. Next, at step 608, the stimulation output is determined via interpolation. At step 610, the evoked response (EMG) for the stimulation output determined at step 608 is measured to determine the target evoked response that is implemented when applying therapy with closed-loop control.

According to the method 620 of FIG. 21, the evoked response itself may be computed at the two threshold values, and the target evoked response is interpolated based on the two thresholds of evoked response. At step 622, stimulation is ramped up, i.e., the pulse width is increased at a constant current amplitude. At step 624, the evoked response (e.g., EMG) is measured at the detection threshold. At step 626, the evoked response (e.g., EMG) is measured at the comfort threshold. At step 628, the target therapy is determined by interpolating between the evoked responses determined at steps 624 and 626.

### System and Method for Real-Time Biological Responses Feedback Based Neural Stimulation Control.

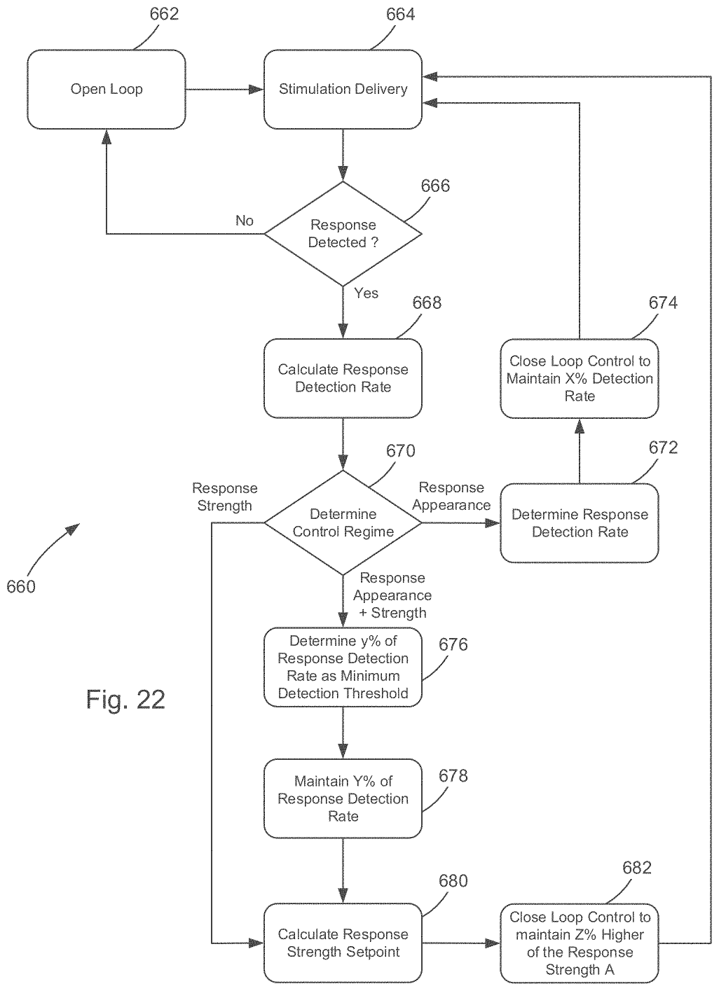

FIG. 22 illustrates a process or method 660 by which to control the application of stimulation therapy. The method 660 can, for example, be implemented with any of the neurostimulator configurations disclosed herein, and can be used to treat any condition or disorder treatable with neural stimulation, such as overactive bladder disorder. While neural stimulation can elicit useful biological responses, some of the evoked biological responses do not share a linear relationship with the provided stimulus. Accordingly, the method 660 implements an algorithm for utilizing the presence and strength of the evoked biological responses, respectively, to control the input stimulus during delivery of therapy.

The method 660 addresses the nonlinearity of the evoked biological responses makes it difficult to use as feedback for controlling for a neural stimulation device. Implementing the method 660, the neurostimulator is adapted to provide effective feedback control during neural stimulation with or without a presence of a biological response. This helps maximize the therapy during application of neural stimulation. The methods 660 utilizes the presence of an evoked biological response, the strength of the evoked response, and voluntary input from the user/subject/patient to modulate the control signal in a closed-loop stimulation application.

Biological responses are not always linear with provided stimulation: higher stimulation doesn't always generate higher biological responses. “Biological responses,” as used herein, refers to any stimulation evoked biological change, i.e., physiological signals, biochemical responses in the body, biomechanical responses, etc. Accordingly, the algorithms implemented by the method 660 should treat the presence of the biological responses, and the strength of the biological responses separately, and according to the general guidelines:

No biological response—Open loop stimulation control within the tolerable stimulation range.

Biological response evoked—Use the frequency of response appearance within a predefined time window as the therapy level, i.e., within a 1 second time window. The appearance of the evoked biological responses should be at least 50% among all the stimulus delivered.

Biological response evoked—Identify the presence of the response, calculate the strength of the response, set x % (include 0%) higher of this strength level as the default therapy level. Patient or physician can set new strength level as the therapy level as needed.

Combine multiple types of biological responses.

Based on the user/subject/patient subjective feelings, voluntary input to control the delivery of neural stimulation can be given, i.e.:

Intentional voluntary input:

User input commands through a device hardware interface or software application, i.e. a physical button pressing on the device, or command input from the app.

a voice command.

Unintentional voluntary input:

User voluntarily generate artifact, noise or voluntary biological response (e.g. from wincing in pain) that manifests in the recording sensors.

User voluntary verbal response (e.g., shout, scream) of the unpleasant stimulation. The device recognize its using its built-in microphone and voice recognition technology.

FIG. 22 illustrates a high level flow chart to show that illustrates the method 660, which functions according to the principles described above. The algorithm implemented by the method 660 is based on the appearance and/or strength of the biological response to the application of stimulation signals. The method 660 uses the appearance and strength of these biological responses as control features in applying closed-loop neurostimulation.

The method 660 can be implemented by a neurostimulator, which applies stimulation therapy via one or more stimulation electrodes, and monitors a biological response, such as an EMG response, via one or more receiving electrodes. The method 660 can, for example, be implemented in any of the neurostimulators disclosed herein.

At step 664, stimulation therapy is delivered via a neurostimulator. At step 666, a determination is made as to whether a response, such as an EMG response, is detected. If no response is detected, the method 660 proceeds to step 662, where the neurostimulation is delivered in open-loop control, i.e., without feedback. The method 660 reverts back to step 664, where stimulation therapy is delivered, and continues to step 666 to determine whether a response is detected. As long as there is no detected response to the stimulation, the method 660 continues to deliver stimulation therapy under open-loop control.

At step 666, if a response, such as an EMG response, to the stimulation is detected, the method 660 proceeds to step 668, where the response detection rate is calculated, then to step 670 where the control regime is determined based on the detection rate. The control regime can be response appearance control, response strength control, or response appearance+strength control. Under response appearance control, the method 660 proceeds from step 670 to step 672 where a determination is made as to the response detection rate that will be the setpoint for closed-loop control. The method 660 proceeds to step 674 where closed-loop control of the stimulation is performed to maintain the X % of the detection rate determined in step 672, where X can be 100 or less. Stimulation parameters, i.e., current amplitude and/or pulse width, are modulated to maintain the detection rate identified in step 672.

Under response strength control, the method 660 proceeds from step 670 to step 680, where a response strength setpoint is calculated. This setpoint is used for closed-loop control. The method 660 proceeds to step 682 where closed-loop control of the stimulation is performed to maintain the response strength at a certain level, Z % greater than the response strength setpoint calculated in step 672, where Z can be zero or greater. Stimulation parameters, i.e., current amplitude and/or pulse width, are modulated to maintain the response strength at the setpoint.

Under response appearance+strength control, the method 660 proceeds from step 670 to step 676, where Y % of the response detection rate is determined as the minimum detection threshold, where y can be 100 or less. At step 678, the minimum detection threshold is used as a setpoint to maintain Y % of the response detection rate under closed-loop stimulation control. The method 660 proceeds to step 680, where a response strength setpoint is calculated. This setpoint is implemented in closed-loop stimulation control at step 682, where the control is performed to maintain the response strength at the certain level, Z % greater than the response strength setpoint calculated in step 672, where Z can be zero or greater. Thus, under the response appearance+strength control scheme, stimulation is modulated under closed-loop control to maintain both a response detection rate and a response strength.

### Use of Informatics for Improving Stimulation Therapy and Patient Outcomes

Referring to FIG. 23, the system can implement a method 640 by which the neurostimulator can be used to provide information that is used to improve stimulation therapy and patient outcomes. According to the method 640, the neurostimulator records information at step 642 and provides this information wirelessly, e.g., via Bluetooth 644, to a patient controller, such as a smartphone or tablet. The information/data is then transmitted via Wi-Fi 648 (local and/or cellular/LTE) and stored on the cloud/server 650. From there, data analysis and informatics are used to determine optimized therapy 652.

The data used at step 652 can be recorded stimulation history, the elicited muscle responses, and the effect the stimulation had on the patient. For example, an overactive bladder patient can use the controller to record a bladder diary that forms a portion of the information/data at step 646. As such, the data transmitted to the cloud/server 650 can include a real-time stimulation history or a quantitative summary of each therapy session.

Once this information is uploaded and available, a portal uses informatics to correlate the three main characteristics: the stimulation profile (e.g., current amplitudes, voltages, pulse profiles), the feedback history (e.g., EMG data), and the patient diaries. The algorithms implemented at the informatics stage 652 use this data to assess the effect of stimulation on the feedback signal and system efficiency. As this data is collected over a larger period of time and over a larger population of patients, it can be used for monitoring patient compliance, usability and efficacy. This information can be used to optimize therapy for each individual patient and thus improving patient outcomes.

### Bilateral Neurostimulation

According to another example implementation of the system, method, and apparatus disclosed herein, an electrical neurostimulation system can include a bilateral stimulation system that implements a garment with a bilateral electrode configuration that fits both right and left feet. Current designs of foot-worn electrical neurostimulation garments distinguish between left and right foot. These designs are not optimal as they require a separate design and manufacturing process for the different garments.

The garment utilized to implement the bilateral stimulation system, method, and apparatus can have various configurations. The garment can, for example, have a brace configuration similar or identical to that shown in FIG. 11 and described herein with reference thereto. As another example, the garment can have a strap configuration similar or identical to that shown in FIGS. 2 and 12 and described herein with reference thereto. As a further example, the garment can have a brace configuration similar or identical to that shown in FIG. 26.

FIG. 26 illustrates an example configuration of a wearable neurostimulator 700 used to implement bilateral neurostimulation. The neurostimulator 700 is generally similar in design and operation to the other brace-type neurostimulators disclosed herein, such as the neurostimulator 110 of FIGS. 3A-4D or the neurostimulator of FIG. 11, with the exceptions described below. The neurostimulator 700 has a brace configuration including a brace 702 upon which the neurostimulator components are supported. The configuration of the neurostimulator 700 is similar in some respects, and identical in others, to the braced configuration shown in FIGS. 3A-4C. The neurostimulator 700 can thus be worn as a garment in the manner shown in FIGS. 3A-B.

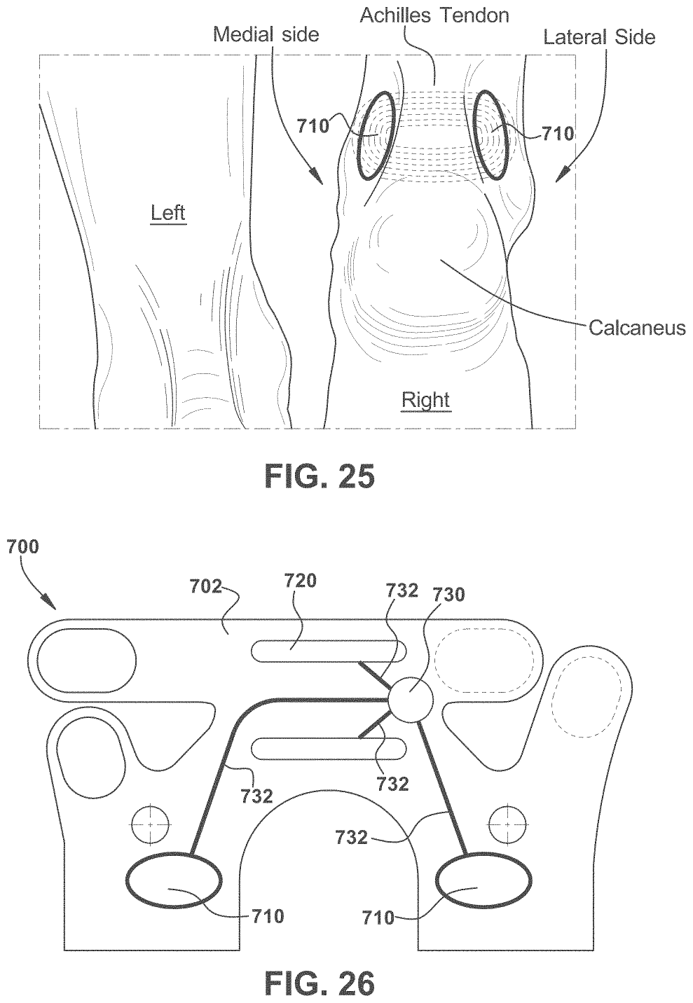

The manner in which the neurostimulator 700 is supported on the subject, i.e., worn, can vary. For example, the neurostimulator could be configured in the form of a sock that fits over the subject's foot and ankle, or in the form of a sleeve that slides over the foot/ankle, leaving the toes exposed. The support structure for positioning the neurostimulator components on the subject can have any configuration suited to place the components at the desired location on the subject, as shown in FIG. 25.

The neurostimulator 700 includes stimulation electrodes 710 and recording electrodes 720. The recording electrodes can be configured and arranged in any of the manners disclosed with reference to any of the configurations disclosed herein. In the example configuration of FIG. 26, the recording electrodes can be configured and arranged in a manner that is similar or identical to that shown and described with reference to FIG. 11. Electrical traces 732 connect the electrodes 710, 720 to the controller 730 or to a connector that is connectable to the controller.

The stimulation electrodes 720 of the neurostimulator 700 have a bilateral electrode configuration that enables bilateral neurostimulation of the target peripheral nerve with a garment that can fit and accommodate right foot and left foot implementations. The neurostimulator 700 can implement any of the closed-loop and/or open-loop neurostimulation control schemes disclosed herein, along with any of the specific features, such as right/left foot detection. This configuration is advantageous in that it allows for a one-device-fits-all approach. Advantageously, this also reduces the design, testing, and manufacturing complexities associated with specific foot configurations.

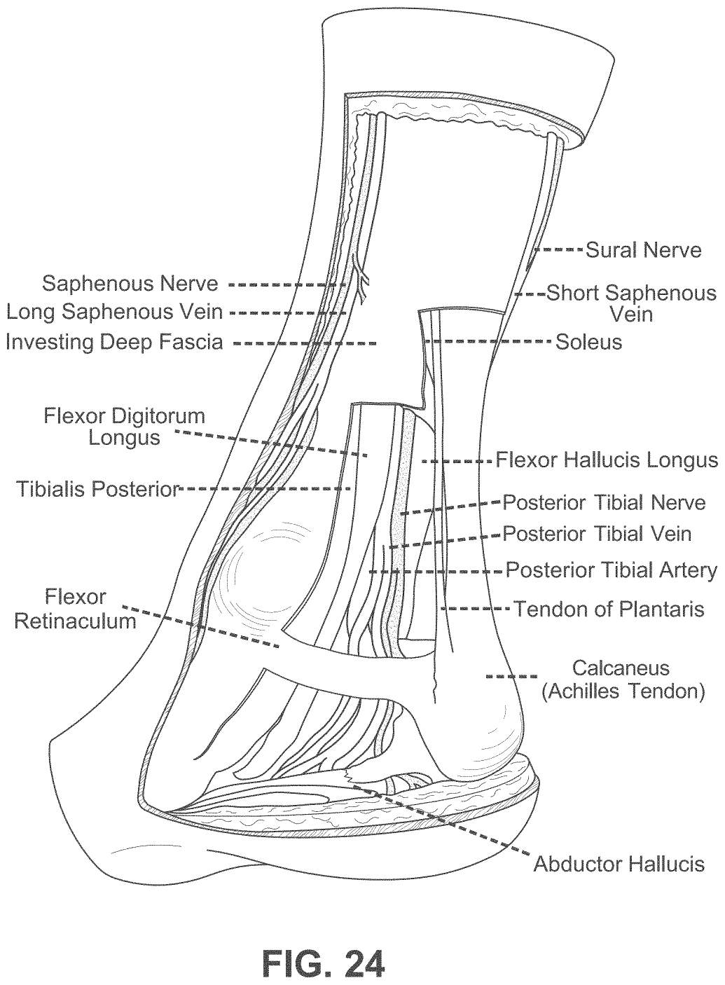

Referring to FIG. 24, the cavity between the Achilles tendon and tibia and fibula bone can be filled with subcutaneous fat, a small amount of body fluid, and other tissues. The posterior tibial nerve lies posterior to the tibial bone and anterior to the Achilles tendon, in the medial-anterior region of this cavity, as shown in FIG. 24.

Current garment designs include two stimulation electrodes on one side of the ankle. These two electrodes, along with a connector work together to apply stimulation to activate the tibial nerve. If the garment is being used on another foot, the electrode configuration needs to be reversed, with the two electrodes and connector on the opposite side of the garment. This is referred to herein as an “ipsilateral design.” For example, the neurostimulator 520 of FIG. 11 is configured for ipsilateral stimulation, with a pair of stimulating electrodes 530 for left foot stimulation, and a pair of stimulating electrodes 532 for right foot stimulation.

The bilateral neurostimulation system, method, and apparatus disclosed herein and implemented via the neurostimulator 700 of FIG. 26 implements neurostimulation electrodes 710 located on both sides of the ankle, medial and lateral, that are connected to a controller via traces 732 and controller 730. This design, which allows the neurostimulation garment to be worn on either the left or the right foot, is referred herein as a “contralateral” design. It should be noted here that referring to the stimulation electrodes 710 on each side of the ankle, it is meant that one or more electrodes are located on each side of the ankle—one or more on the medial side, and one or more on the lateral side. Thus, the stimulation electrodes 710 can be series or arrays of electrodes on each side of the ankle, with the selection and operation of individual electrode pairs being performed in a manner consistent with the methods described herein.

The bilateral design of the neurostimulator 700, when worn, produces a contralateral positioning of the stimulation electrodes 710, i.e., positions the stimulation electrodes on opposite sides of the foot/ankle, in the manner shown in FIG. 25. As shown, the stimulation electrodes are positioned on opposite sides of the cavity between the Achilles tendon and tibia and fibula bone, adjacent the medial-anterior region of the cavity where the posterior tibial nerve lies (see, FIG. 24). The stimulation electrodes 710 are therefore positioned on opposite sides of the tibial nerve. Positioned as such, the stimulation electrodes 710 produce stimulation pulses laterally across the cavity and across the tibial nerve.

The electrical field created between the stimulation electrodes 710 in the contralateral electrode configuration of the bilateral design extends across the foot, i.e., from the medial side to the lateral side (or vice versa, depending on how the electrodes are energized), as indicated generally by the flux lines (dashed lines) shown in FIG. 25. As such, the stimulation field and the energy applies to the tibial nerve acts perpendicular to the axon fibers in the tibial nerve. This is opposed to an ipsilateral electrode configuration in which the electrical stimulation field and the flux lines produced by the field, which extend/flow more parallel to the axon fibers of the tibial nerve. As a result, the contralateral configuration of the stimulation electrodes 710 produces an electrical stimulation field that penetrates deeper through the tissue, thereby increasing the probability that axon fibers in the tibial nerve will be recruited.

The fact that the bilateral design of the neurostimulator 700 implementing the contralateral electrode configuration produces an electrical stimulation field that penetrates across the foot and thereby helps ensure tibial nerve recruitment provides some liberty in selecting the configuration and operation of the stimulation electrodes 710. An example is the identification/selection of which of the stimulation electrodes 710 is the anode and which is the cathode. The bilateral positioning of the stimulation electrodes 710 helps ensure that the tibial nerve fibers will be activated regardless of which side is the anode and which side is the cathode. Therefore, the neurostimulator 700 can apply anodic or cathodic stimulation to the tibial nerve from either side of the nerve, which lends flexibility to the system. This also allows for determining which method of application renders the most effective stimulation results.

A stimulating electrode can act as an anode (+, source of current) or a cathode (−, sink of current). Experimental data has shown that cathodic stimulation elicits neural responses (action potentials) more readily than anodic stimulation. Given the same stimulation strength or energy, cathodic stimulation elicits neural responses more readily than anodic stimulation. The power level (amplitude) required to elicit such a response through anodic stimulation can, for example, be several times that required of cathodic stimulation.

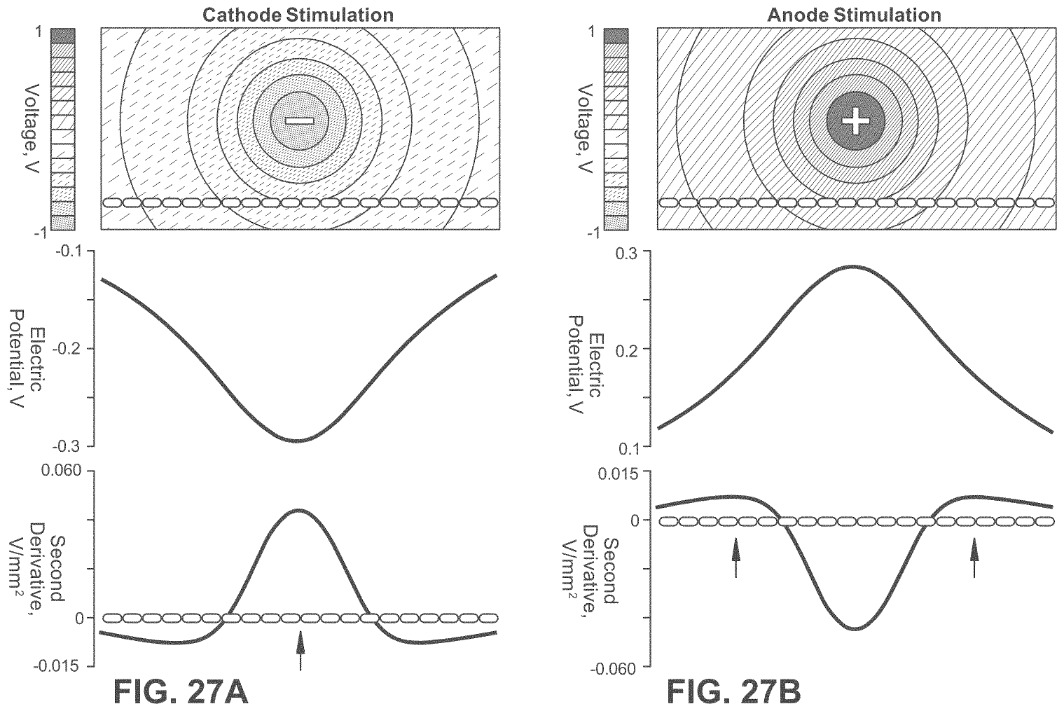

The effectiveness of a stimulation signal in recruiting a nerve is indicated or related to the second derivative of electrical potential of the signal. This is shown in FIGS. 27A (cathodic stimulation) and 27B (anodic stimulation). As shown in these figures, the second derivative for cathodic stimulation has a peak magnitude, indicated generally at the arrow in FIG. 27A that is significantly higher than that for anodic stimulation, which is indicated generally at the arrows in FIG. 27B. Thus, for a given power level, cathodic stimulation can be more effective at nerve recruitment.

This is not to say that cathodic stimulation is necessarily more desirable than anodic stimulation. Because, however, with all things being equal, cathodic stimulation has been shown to require less power/energy, it has naturally evolved as the primary arrangement. The bilateral configuration of the neurostimulator 700, positioning the electrodes on opposite sides of the foot and the tibial nerve, optimizes the effectiveness of all stimulation fields, cathodic or anodic, because the field acts laterally, as described above, which helps ensure a high probability of nerve recruitment, as long as the strength of the field is sufficient.

As described herein previously, the effects of tibial nerve stimulation depend on the stimulation parameters, i.e., the amplitude, frequency, and waveform characteristics, i.e., pulse parameters, of the stimulation pulses. The effects of tibial nerve stimulation can also depend on properties of the stimulated tissue, such as the distribution and orientation of the nerve axons, tissue, structures, fluids, etc. surrounding the nerve. Because of this, it can be difficult to determine which electrode configurations and/or arrangements will be most effective. As a result, determining the electrode configurations and/or arrangements that prove to be most effective for a particular subject can be a matter of trial-and-error. Advantageously, the bilateral design of the neurostimulator 700 facilitates identifying the ideal configuration/arrangement of the stimulation electrodes 710 because the arrangement helps ensure that stimulation energy will reach the tibial nerve.

The bilateral design of the neurostimulator 700 allows it to have no assigned role for anode and cathode. The controller 730 will assign the cathode/anode identity, which can be switched back and forth. Thus, for example, during one stimulating pulse, one stimulation electrode 710 can be the cathode positioned medially and the other the anode positioned laterally. During next stimulating pulse, the cathode/anode designation can be reversed. This cathode/anode electrode switching can be done in any pattern and order, e.g., every pulse, every other pulse, every five pulses, etc.

Because the neurostimulator 700 is ambidextrous and can fit the right or left foot, the cathode/anode identifications for the stimulating electrodes 710 varies depending on the foot upon which the neurostimulator is worn. One example configuration, the identity of the foot—right or left—can be determined automatically, as described above. In another example configuration, the user can select which foot the stimulation is being applied. If, for example, the device is used on the right ankle, user will selected the ‘Right Foot’ on the patient interface, then one stimulation electrode 710, medial or lateral, can be the cathode and the other can be the anode. The bilateral design of the neurostimulator 700 is therefore advantageous because it is designed for both feet, and the user doesn't need to 1) purchase two devices and 2) be concerned with which foot the device is worn upon.

Additionally, the bilateral construction and contralateral operation of the neurostimulator 700 can be less complicated because the stimulation electrodes 710 on both sides are used in every instance of operation. Advantageously, the neurostimulator 700 does not require any switching between right/left electrode pairs, such as in the example configuration of FIG. 11, because both sides are used. In the configuration of FIG. 11, one pair of stimulation electrodes on one side of the foot/ankle/leg is used, depending on the foot upon which the neurostimulator is worn, with the other being idle. This active/idle designation of electrode pairs requires switching, which further complicates the design. For the bilateral neurostimulator 700, implementing the contralateral stimulation electrode configuration with both electrodes 710 being used in every instance, this active/idle switching is avoided. The electrical configuration of the neurostimulator 700 of FIG. 26 is therefore simplified over the neurostimulator 520 of FIG. 11, as evidenced, for example, by the simplicity of the electrical traces 732 of FIG. 26, as compared to the traces 534 of FIG. 11. At the same time, this simplified design can provide improved stimulation performance.

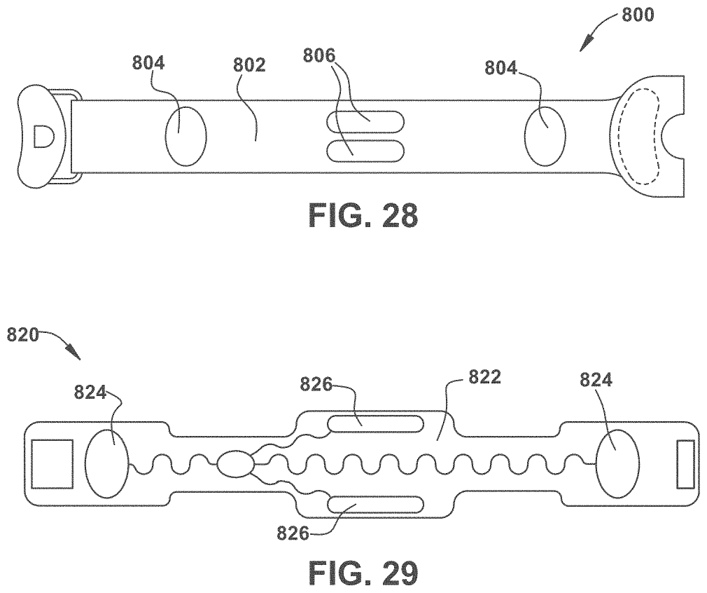

FIG. 28 illustrates a strap implementation of the neurostimulator 800 that is similar to the strap implementation of FIGS. 2A-2E. The neurostimulator 800 can be similar or identical in any or all respects to the neurostimulator of FIGS. 2A-2E, so the description of that implementation applies to that of FIG. 28 where applicable, the difference being the implementation of bilateral stimulation electrodes in the configuration of FIG. 28. As shown in FIG. 28, the neurostimulator 800 includes a strap 802 that supports stimulation electrodes 804 arranged in a bilateral fashion. The neurostimulator 800 also includes recording electrodes 806 positioned centrally on the strap 802. The neurostimulator 800 can be worn in the manner shown in FIGS. 1A-1B, which positions the stimulation electrodes in the bilateral positions described above with reference to FIGS. 24 and 25. The neurostimulator 800 operates in a manner consistent with that described above with reference to the implementation of FIG. 26.

FIG. 29 illustrates a strap implementation of the neurostimulator 800 that is similar to the strap implementation of FIG. 12. The neurostimulator 800 can be similar or identical in any or all respects to the neurostimulator of FIG. 12, so the description of that implementation applies to that of FIG. 29 where applicable, the difference being the implementation of bilateral stimulation electrodes in the configuration of FIG. 29. As shown in FIG. 29, the neurostimulator 820 includes a strap 822 that supports stimulation electrodes 824 arranged in a bilateral fashion. The neurostimulator 820 also includes recording electrodes 826 positioned centrally on the strap 802. The neurostimulator 800 can be worn in the manner shown in FIGS. 1A-1B, which positions the stimulation electrodes in the bilateral positions described above with reference to FIGS. 24 and 25. The neurostimulator 820 operates in a manner consistent with that described above with reference to the implementation of FIG. 26.

While aspects of this disclosure have been particularly shown and described with reference to the example aspects above, it will be understood by those of ordinary skill in the art that various additional aspects may be contemplated. A device or method incorporating any of the features described herein should be understood to fall under the scope of this disclosure as determined based upon the claims below and any equivalents thereof. Other aspects, objects, and advantages can be obtained from a study of the drawings, the disclosure, and the appended claims.

We claim:

1. An apparatus for applying electrical stimulation to a target peripheral nerve in a subject, comprising:
a wearable structure configured to be worn on at least one of the subject's lower leg, foot, or ankle;
a first stimulation electrode mounted on the wearable structure at a first location on the wearable structure;
a second stimulation electrode mounted on the wearable structure at a second location on the wearable structure, different than the first location on the wearable structure;
one or more recording electrodes mounted on the wearable structure; and
a control unit for controlling the operation of the first stimulation electrode, the second stimulation electrode, and the one or more recording electrodes;
wherein the wearable is configured to position the first stimulation electrode on a medial side of the target peripheral nerve, and to position the second stimulation electrode on a lateral side of the target peripheral nerve;
wherein the control unit is configured to use the first and second stimulation electrodes to apply electrical stimulation to the target peripheral nerve and to record physiological responses to the applied electrical stimulation using the recording electrodes.

2. The apparatus recited in claim 1, wherein the controller is configured to apply the electrical stimulation to the target peripheral nerve using one of the first and second stimulation electrodes as a cathode and the other of the first and second stimulation electrodes as an anode.

3. The apparatus recited in claim 2, wherein the controller is configured to apply cathodic electrical stimulation to the target peripheral nerve.

4. The apparatus recited in claim 2, wherein the controller is configured to apply anodic electrical stimulation to the target peripheral nerve.

5. The apparatus recited in claim 2, wherein the controller is configured to switch between applying cathodic and anodic electrical stimulation to the target peripheral nerve.

6. The apparatus recited in claim 1, wherein the target peripheral nerve is the tibial nerve and wherein the wearable is configured to position the first stimulation electrode medially of the tibial nerve, and to position the second stimulation electrode laterally of the tibial nerve.

7. The apparatus recited in claim 6, wherein the controller is configured to apply electrical stimulation laterally across the tibial nerve in a direction that acts perpendicular to the axon fibers in the tibial nerve.

8. The apparatus recited in claim 6, wherein the wearable is configured to position the first stimulation electrode on a skin surface of the subject located on a medial side of a cavity between an Achilles tendon, tibia bone, and fibula bone of the subject through which the tibial nerve extends, and to position the second stimulation electrode on a skin surface of the subject located on a lateral side of the cavity.

9. The apparatus recited in claim 8, wherein the controller is configured to apply electrical stimulation laterally across the cavity from the medial side to the lateral side and vice versa.

10. The apparatus recited in claim 1, further comprising electical traces secured to the wearable structure, wherein the electrical traces are configured to electrically connect the stimulation electrodes and recording electrodes to the control unit.

11. The apparatus recited in claim 1, wherein the control unit is configured to detect via the recording electrodes the presence of an EMG response to stimulation therapy, and the control unit is further configured to:
in response to detecting no EMG response, deliver stimulation therapy under open-loop control without EMG feedback; and
in response to detecting an EMG response, deliver stimulation therapy under closed-loop control with EMG feedback.

12. The apparatus recited in claim 1, wherein the control unit is configured to detect via the stimulating electrodes whether the apparatus is being worn on a right foot or left foot of the user and, in response to detecting the foot, is further configured to determine which stimulation electrode is configured as a cathode and which stimulation electrode is configured as an anode.

13. The apparatus recited in claim 1, wherein the wearable comprises an ankle brace having a first portion configured to be strapped around a foot and to position the one or more recording electrodes on the foot, and a second portion configured to be strapped around an ankle to position the first stimulation electrode on a medial side of the target peripheral nerve, and to position the second stimulation electrode on a lateral side of the target peripheral nerve.

14. The apparatus recited in claim 1, wherein the wearable comprises a strap having a first portion configured to be wrapped around a foot and to position the one or more recording electrodes on the foot, and a second portion configured to be strapped around an ankle to position the first stimulation electrode on a medial side of the target peripheral nerve, and to position the second stimulation electrode on a lateral side of the target peripheral nerve.
